(19) United States
(12) Patent Application Publication
(10) Pub. No.: US 2009/0193532 A1
(43) Pub. Date: Jul. 30, 2009

(54) Genetically Engineered and Phenotyped Mice and Stem Cell Clones for Producing the Same

(76) Inventors: Alejandro Abuin, The Woodlands, TX (US); Joel A. Edwards, Las Flores, CA (US); Charles Montgomery, Jay, OK (US); Carolina Rangel, Houston, TX (US); Arthur T. Sands, The Woodlands, TX (US); Zheng-Zheng Shi, The Woodlands, TX (US); Mary Jean Sparks, Magnolia, TX (US); Peter Vogel, The Woodlands, TX (US); Brian Zambrowicz, The Woodlands, TX (US)

Correspondence Address: LEXICON PHARMACEUTICALS, INC., 8800 TECHNOLOGY FOREST PLACE, THE WOODLANDS, TX, 77381-1160, US

(21) Appl. No.: 12/099,459

(22) Filed: Apr. 8, 2008

Related U.S. Application Data
(63) Continuation of application No. 11/334,806, filed on Jan. 18, 2006, now abandoned.
Application No. 11/334,806 is a continuation-in-part of application No. 10/843,704, filed on May 11, 2004, now abandoned.
Application No. 10/843,704 is a continuation of application No. 09/963,299, filed on Sep. 26, 2001, now abandoned.
Application No. 11/334,806 is a continuation-in-part of application No. 10/875,104, filed on Jun. 23, 2004, now abandoned.
Application No. 10/875,104 is a continuation of application No. 09/880,711, filed on Jun. 12, 2001, now abandoned.
Application No. 11/334,806 is a continuation-in-part of application No. 10/935,709, filed on Sep. 7, 2004, now abandoned.
Application No. 10/935,709 is a continuation of application No. 10/060,069, filed on Jan. 29, 2002, now abandoned.
(60) Provisional application No. 60/237,272, filed on Oct. 2, 2000.
(60) Provisional application No. 60/211,230, filed on Jun. 12, 2000.
(60) Provisional application No. 60/265,585, filed on Jan. 31, 2001.

(51) Int. Cl. A01K 67/027

(52) U.S. Cl. 800/18

(57) ABSTRACT

The current invention relates to genetically engineered mice, cells derived from those mice, and polynucleotides and polypeptides corresponding to genes affected by the engineered mutation. The invention also relates to antibodies raised in a mouse of the invention. The invention further provides methods for using the mice, cells, polynucleotides, polypeptides and antibodies of the invention.

This application is a continuation-in-part of U.S. application Ser. No. 10/843,704 filed May 11, 2004, currently pending, which is a continuation of U.S. application Ser. No. 09/963,299 filed Sep. 26, 2001, now abandoned, which claims the benefit of U.S. Provisional Application Ser. No. 60/237,272 filed Oct. 2, 2000, now abandoned; U.S. application Ser. No. 10/875,104 filed Jun. 23, 2004, currently pending, which is a continuation of U.S. application Ser. No. 09/880,711 filed Jun. 12, 2001, now abandoned, which claims the benefit of U.S. Provisional Application Ser. No. 60/211,230 filed Jun. 12, 2000, now abandoned; U.S. application Ser. No. 10/935,709 filed Sep. 7, 2004, currently pending, which is a continuation of U.S. application Ser. No. 10/060,069 filed Jan. 29, 2002, now abandoned, which claims the benefit of U.S. Provisional Application Ser. No. 60/265,585 filed Jan. 31, 2001, now abandoned; all of which are incorporated herein by reference.

### 1.0 SUBMISSION ON COMPACT DISC

The contents of the following submission on compact discs are incorporated herein by reference in their entirety: a compact disc copy of the Sequence Listing (COPY 1) (file name: Lexicon 3423 Listing.txt; date recorded: Jan. 16, 2006; size: 1,380 kilobytes); a duplicate compact disc copy of the Sequence Listing (COPY 2) (file name: Lexicon 3423 Listing.txt; date recorded: Jan. 16, 2006; size: 1,380 kilobytes); and a computer readable form copy of the Sequence Listing (CRF COPY) (file name: Lexicon 3423 Listing.txt; date recorded: Jan. 16, 2006; size: 1,380 kilobytes).

### 2.0 FIELD OF THE INVENTION

The present invention relates to genotypic and phenotypic analyses of multiple genes in mice for the rapid and efficient development of drugs and therapies. The invention also relates to the individual mutant animal lines and their respective phenotypes, ES cells to generate animals and other cell lines, and antibodies generated in the animals. The massive phenotypic analysis provided by the present invention represents a key breakthrough in making drug development for therapy more efficient and thus faster and more affordable.

### 3.0 BACKGROUND

The development of new drugs and therapies has traditionally benefited from modeling the disease of interest, typically in a suitable animal species. Species of particular interest share a close evolutional relationship to humans, are physiologically relevant to human disease, are capable of supporting research through short generational cycles, and are scientifically well defined to generate consistent and meaningful data of disease phenotype and physiology of drug effects. For over a century, mice have been recognized as highly suited for drug development.

Where animal models for disease are sought, they can be obtained by observing phenotypes in animals to find a naturally occurring variant that mimics the pathology of a disease of interest as closely as possible. Ideally, such a variant would be the result of a naturally occurring mutation so that the animal could be propagated for research. Yet, the likelihood of finding such animal models is small and only a limited number of desirable animal models for diseases of interest have been identified this way.

New ways of generating mouse variants resulted from technologies for engineering mutations into the genome of mice. These technologies facilitate the introduction of changes into the mouse genome, either through random or targeted events. Of particular value is site specific mutagenesis of the mouse genome, especially when combined with the genetic and phenotypic characterization of the resulting mouse variant. Site specific mouse mutants have provided valuable insights that have been key in improved allocation of drug development efforts and thus saved valuable time und ultimately lives.

Expanding the number of mouse variants with engineered depletion of genomic function would provide additional opportunities for more efficient drug development for therapy. Particularly desirable would be a bank of mouse mutants, each representing a depletion of a particular genomic function, each being characterized as to the specific site of the mutation and the gene affected, and each mutant being characterized on the homozygote, heterozygote, and wild-type level for a number of different physiological parameters for quick identification of most desirable phenotypes for multiple types of diseases. Such a bank of mouse mutants would be especially useful if it included functional depletions for most genes in the mouse genome as that would substantially increase the probability of obtaining a mouse mutant with a phenotype specific for many diseases of interest.

The present invention provides such a bank of mouse mutants in certain embodiments. More specifically, the present invention provides a bank of multiple mutant mouse lines, representing functional depletions of many genes of the mouse genome, all genetically and phenotypically characterized.

### 4.0 SUMMARY OF THE INVENTION

The present invention provides in certain embodiments a library or bank of mice (mouse library), each mouse comprising a mutation engineered into its genome, and in certain preferred embodiments, each mouse of the library comprises an engineered mutation that is distinct from the mutation engineered into the other mice of the library (distinct engineered mutation). In certain preferred embodiments, a mouse library according to the invention comprises at least 50 mice with each carrying a distinct engineered mutation, and more preferably at least 100 mice, 1,000 mice, 10,000 mice, or 50,000 mice, each library comprising mice carrying a distinct engineered mutation. The current invention also comprises individual mice and mouse lines carrying an engineered mutation.

In certain preferred embodiments, a mouse library of the current invention comprises mice that can be bred to be heterozygous and/or homozygous for the engineered mutation. In certain other preferred embodiments, a mouse library of the current invention comprises mice that have been characterized as to the engineered mutation, changes in gene expression, and the phenotype of the mice, preferably heterozygous and homozygous for the engineered mutation. In certain embodiments, the current invention provides animal models for drug development. For example, a mouse of the current invention, in certain embodiments, lacks a genomic function and said function is linked to phenotypic traits affiliated with one or more diseases.

The invention further comprises cells, for example, an embryonic stem cell (ES cell), a fibroblast, a nerve cell, a muscle cell, any cell derived from an ES cell of the invention. In certain embodiments, a cell of the invention carries an engineered mutation.

In certain other embodiments, the current invention provides antibodies, and in certain preferred embodiments, antibodies generated using a mouse of the invention.

### 5.0 BRIEF DESCRIPTION OF THE DRAWINGS

FIGS. 1-18: Each figure presents one of gene trap vectors VICTR21, VICTR22, VICTR23, VICTR24, VICTR25, VICTR30, VICTR31, VICTR32, VICTR37, VICTR40, VICTR41, VICTR44, VICTR48, VICTR48 MTII, VICTR49, VICTR54, VICTR628, and VICTR743. The total length of each vector is provided in nucleotides. Abbreviations followed by an arabic number represent restriction enzymes and the number constitutes the number of the nucleotide in the vector at which the enzymes are capable of cutting the vector. Enzymes with an asterix at the number can cut the vector only at a single site. Non-cutters are enzymes that cannot cut the vector at any site. Sequence elements of the vector are abbreviated as follows. Where a sequence element is written upside down, it means the orientation of the element is reversed. LTR is a modified (deleted enhancer) long terminal repeat from Moloney murine leukemia virus; SA is a splice acceptor; SA-TM is a splice acceptor-transmembrane domain for trapping secreted proteins more effectively (allowing the fusion protein resulting from genomic and vector sequences to be retained intracellularly); frt is FRT recombinase; IRES is an internal ribosome entry site from EMCV (encephalomyocarditis virus); BGEO (or βGEO) is a fusion of betagalactosidase and neomycin (neo) resistance gene; pA is a SV40 gene polyadenylation signal sequence; PGK is a phosphoglycerate kinase promoter from mouse phosphoglycerate kinase-1 gene; BTK is a mouse Bruton agammaglobulinemia tyrosine kinase gene, first non-coding exon; PURO is a puromycin resistance gene coding sequence that lacks a polyadenylation sequence; SD is a splice donor sequence from mouse BTK first exon; NEO is a neomycin resistance gene; Gastrin is the first exon of the gastrin gene; SV40tpA is a concatenated triple polyadenylation signal from the major viral coat protein 1 gene of the SV40 virus; BGlobinTerm is a transcriptional terminator that operates in opposite direction of viral transcription; CRE is cre recombinase; MTII pA is a 530 base pair segment that includes the last two exons, with intervening intron, of the mouse metallothionein 2 gene and its polyadenylation signal.

### 6.0 DETAILED DESCRIPTION OF THE INVENTION

The present invention describes libraries of mice carrying engineered mutations affecting multiple genes and facilitating efficient drug development for therapy. The present invention provides mouse libraries, individual lines of mice comprising the described mouse libraries, ES cells to generate mice and other cell types specific for the mice of the libraries. The invention also comprises other compositions and methods related to the mouse libraries and ES cells described herein.

### 6.1 Mouse Libraries of the Current Invention

A mouse library of the invention preferably comprises a collection of mice and, more preferably, mice of said collection have an engineered genomic mutation. A collection of mice comprising a mouse library of the invention, in certain embodiments, comprises at least 50 mice, or at least 100 mice, at least 200 mice, at least 300 mice, at least 400 mice, at least 500 mice, at least 750 mice, at least 1000 mice, at least 2500 mice, at least 5000 mice, at least 7500 mice, at least 10000 mice, at least 25000 mice, at least 50000 mice, or at least 100000 mice. The mice in a collection of mice of the invention, in certain preferred embodiments, comprise an engineered genomic mutation. A collection of mice of the present invention, in certain embodiments, comprises at least 50 different engineered genomic mutations, or at least 100, at least 200, at least 300, at least 400, at least 500, at least 750, at least 1000, at least 2500, at least 5000, at least 7500, at least 10000, at least 25000, at least 50000, or at least 100000 engineered genomic mutations.

A mouse of a mouse library of the invention, in certain embodiments, may be of any strain, or of a mixture of strains, or a chimera, or a derivative of one or more strains. Examples of mouse strains are 129, Black 6, C57BL/6, CD-1. See, for example, U.S. Pat. Nos. 6,878,542; 6,815,185 for mouse strains, all of which are incorporated herein by reference for all purposes. A listing of various mouse strains is provided in “Genetic Variants and Strains of the Laboratory Mouse” 3rd Ed., Vols. 1 and 2, 1996, Lyon et al., eds., Oxford University Press, NY, N.Y., herein incorporated by reference in its entirety. The mouse may be a female or male mouse, a young mouse, an adult mouse, a mouse with a known phenotype or an unknown phenotype, a mouse with a known genotype or an unknown genotype.

An engineered mutation in a mouse of a library of the current invention affects the genome in the nucleus of the cells of the mice. An engineered mutation, in certain embodiments, comprises any one, two, three, four, five or more of an insertion, a deletion, a replacement, an inversion, a truncation, a point mutation, a translocation, a duplication, an amplification, a recombination, and/or any other kind of alteration of the genome. A mutation may, in certain embodiments, affect, change, disturb or alter the structure of the genome of a mouse, or the function of the genome, or both. A mutation may affect, change, disturb or alter the genome of a mouse, a chromosome, a gene, a promoter, an enhancer, a telomere, an intron, an exon, a splice site, an untranscribed region, an untranslated region, a transcribed region, a translated region, a transcription initiation site, a translation initiation site, a termination codon, a polyadenylation signal, a centromer, or any other structure or element found in the genome. A mutation may comprise, in certain embodiments, an engineered or recombinant polynucleotide of at least 500 base pairs, at least 1000 base pairs, at least 2000 base pairs, at least 5000 base pairs, at least 10000 base pairs, or at least 20000 base pairs.

An engineered genomic mutation of the current invention, in certain embodiments, comprises a mutation that is caused through human influence or intervention. For example, any human effort that increases the probability of a mutation is contemplated. In certain preferred embodiments, an engineered genomic mutation results from an assay or technique known in the field of biotechnology, molecular biology or genetic engineering, or any other discipline engaged in the manipulation of a mammalian genome. Where a mutation is engineered into the genome of a cell or an animal, off-spring of said cell or animal are also contemplated as carrying said engineered genomic mutation.

A mouse library of the current invention may be maintained, kept or stored, in certain embodiments, as a collection of living animals, a collection of ES cells, a collection of eggs, a collection of fertilized eggs, a collection of gametes, a collection of morulae, a collection of blastocysts, a collection of embryos, a collection of oocytes, a collection of fetuses, or a collection of cells of any cell type or cell aggregate that is capable of generating a mouse. Such a collection would comprise, in certain embodiments, each engineered genomic mutation so that a mouse of each mouse line of the mouse library may be generated. A mouse library of the current invention may be stored, in certain embodiments, through cryopreservation. In certain embodiments, cryopreservation is not used to store a living animal. Methods for cryopreservation useful to store a mouse library of the current invention are known in the art and are described, for example, in U.S. Pat. Nos. 5,962,213; 6,361,934; 6,500,608; 6,503,698; 6,519,954; and in Van den Abbeel et al., 1994, Cryobiology 31(5):423-33; Dumoulin et al, 1994, Fertil Steril. 62(4):793-8; Vasuthevan et al., 1992, J. Assist. Reprod. Genet. 9(6):545-50; Thornton et al., 1999, Mamm. Genome 10(10):987-92; Songsasen and Leibo, 1997, Cryobiology 35(3):255-69; Songsasen and Leibo, 1997, Cryobiology 35(3):240-54; Songsasen et al., 1997, Biol. Reprod. 56(1):143-52; Critser and Mobraaten, 2000, ILAR J. 41(4):197-206; Kasai and Mukaida, 2004, Reprod. Biomed. Online 9(2):164-170; Menezo, 2004, Obstet. Gynecol. Reprod. Biol. 115 Suppl 1:S12-5; Sztein et al, 2001, Cryobiology 42(1):28-39; Sztein et al., 1999, Lab.-Anim.-Sci. 49(1):99-100; Sztein et al., 1998, Biol.-Reprod. 58(4):1071-4, all of which are incorporated herein by reference for all purposes.

Mutations and ways of engineering mutations into a mouse genome are also described in U.S. Patent Application Nos. 20020182724, 20040072243, 20040259253, 20050059060, 20050053953; and in U.S. Pat. Nos. 6,080,576; 6,136,566; 6,207,371; 6,218,123; 6,436,707; 6,776,988; 6,808,921; 6,855,545, all of which are incorporated herein by reference for all purposes.

### 6.1.1 Generating Mouse Libraries of the Current Invention

A mouse library of the current invention, in certain embodiments, is generated by engineering a genomic mutation into a plurality of mice. A genomic mutation is engineered into a mouse, in certain embodiments, by engineering said mutation into a cell, and preferably into a cell that is useful to generate a mouse. In certain embodiments, a mutation is engineered into a gamete, a fertilized egg, a stem cell, an embryonic stem cell, a hematopoietic stem cell, or any other type of cell useful for generating a mouse. Examples of cell lines useful for generating a mouse include AB 2.2, HES-1, J1, CGR-8, R1, E14.1, E14TG2a. See, for example, U.S. Pat. Nos. 6,875,607; 6,867,035; 6,878,542; 6,815,185; 6,884,622 for cell lines, all of which are incorporated herein by reference for all purposes.

A genomic mutation, in certain embodiments, is engineered by introducing an oligonucleotide or a polynucleotide into a cell that is useful to generate a mouse. A useful nucleotide, oligonucleotide, or polynucleotide may be naturally occurring or not naturally occurring. In certain preferred embodiments, DNA, RNA, or any kind of derivative of either one, is introduced into a cell useful to generate a mouse. A DNA or RNA, or derivative of either, useful for introduction into a cell, in certain embodiments, is a vector, a virus, a construct, a replacement vector, an integration vector, a linear molecule, a circular molecule. A polynucleotide useful for engineering a genomic mutation into a cell, in certain embodiments, comprises one, two, three, or more of a marker, a marker facilitating the survival of a cell, a marker facilitating the killing of a cell, a stretch of genomic sequence, a splice donor site, a splice acceptor site, a multi-cloning site, a poly-adenylation signal, a transcription initiation site, a translation initiation site, an intron, an exon, vector sequences or any other element desired for engineering a desired genomic mutation. A stretch of genomic sequence may, in certain embodiments, have a length of 0 to 1 million bases, or 0.5 to 2 million bases, 0.2 to 20.0 kilobases, 0.2 to 5.0 kilobases, or 1.0 to 10.0 kilobases. Examples of vectors are Bluescript, pUC, or any other vector that facilitates the cloning, manipulation, sequencing, identification, isolation, amplification, and/or purification of a polynucleotide. See, for example, U.S. Pat. Nos. 6,815,185; 6,884,622 for background on designing, manipulating and cloning polynucleotides, all of which are incorporated herein by reference for all purposes.

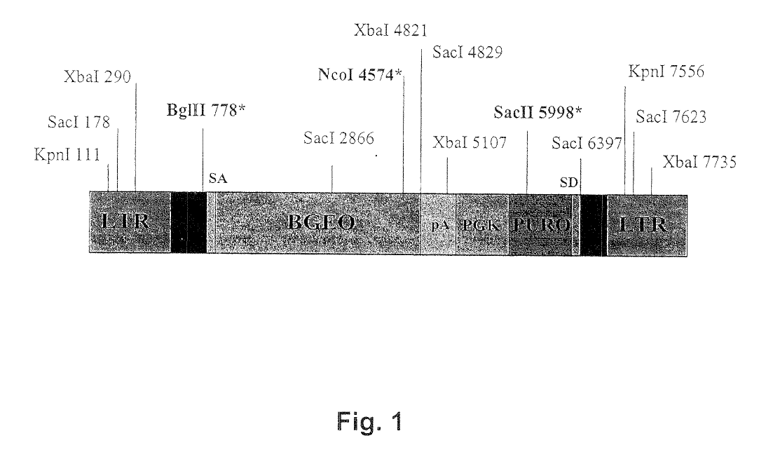

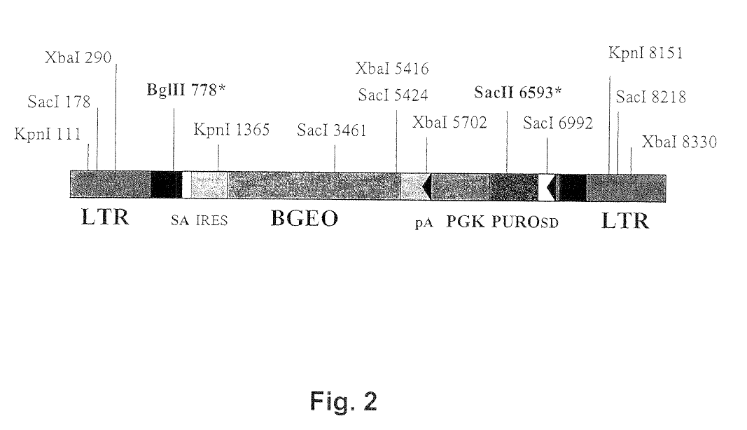

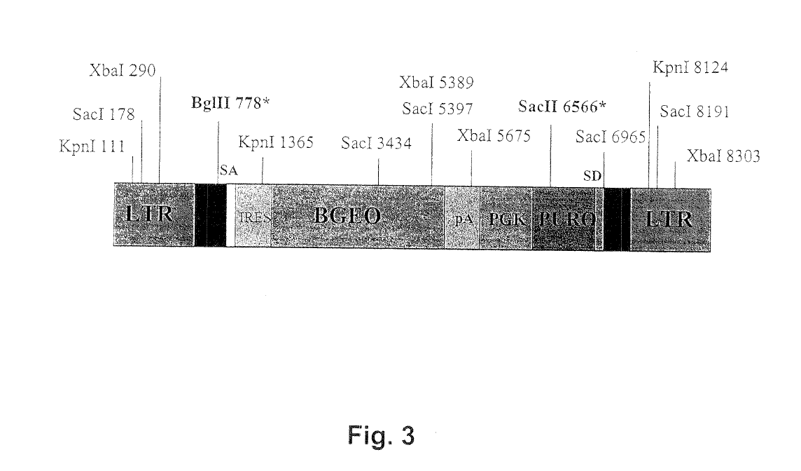

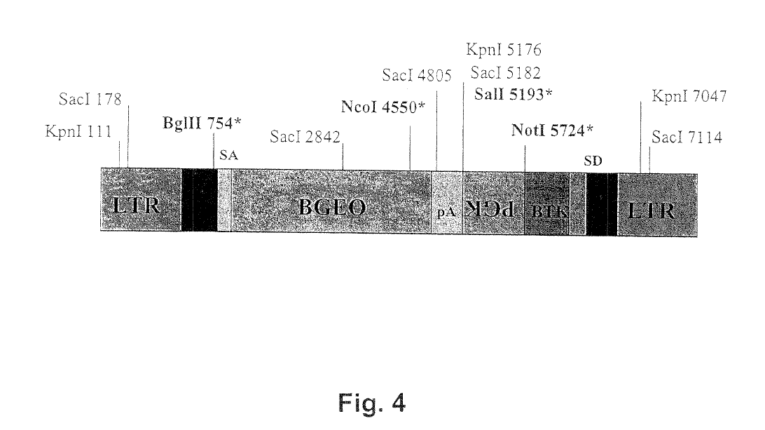

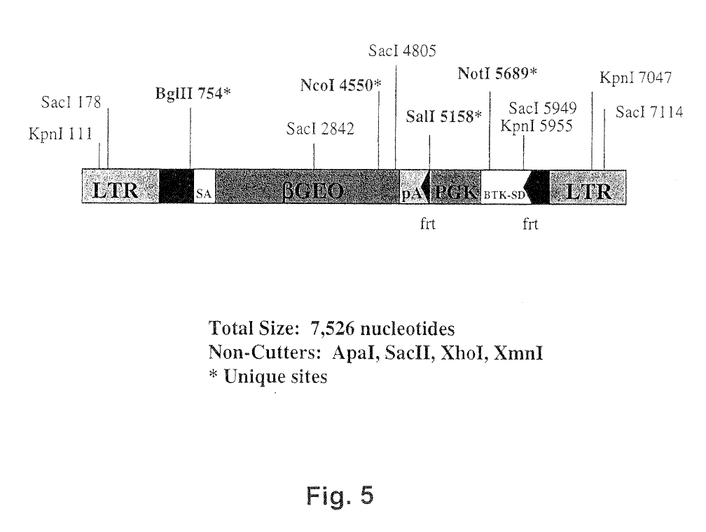

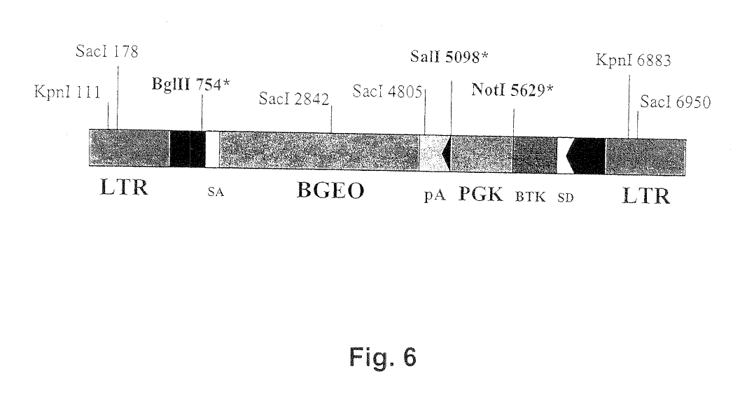

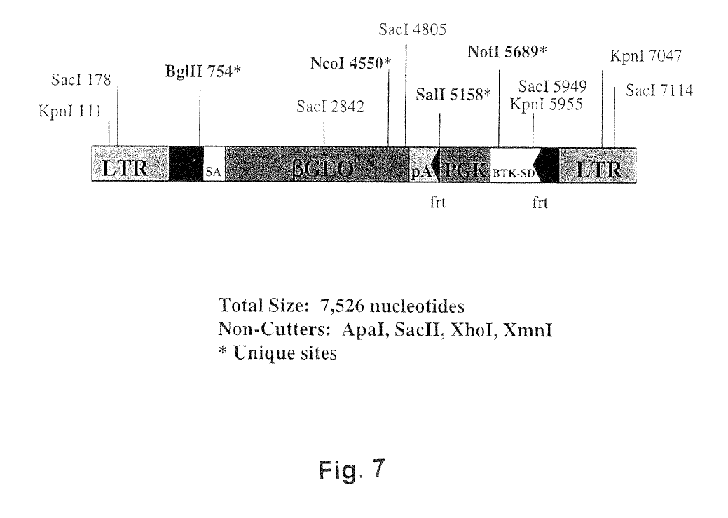

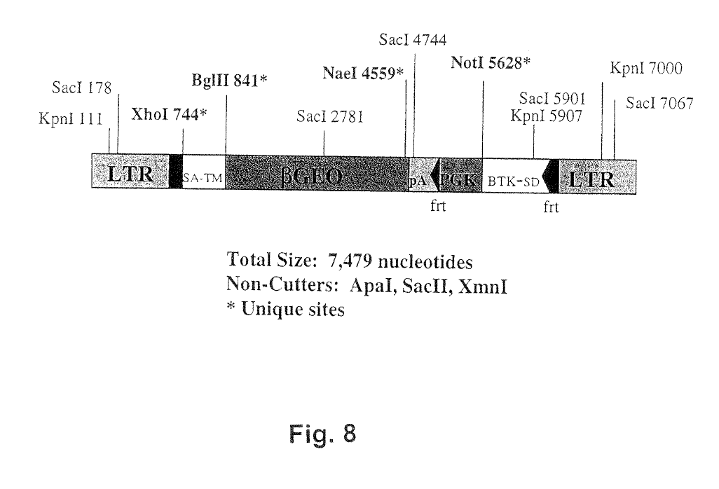

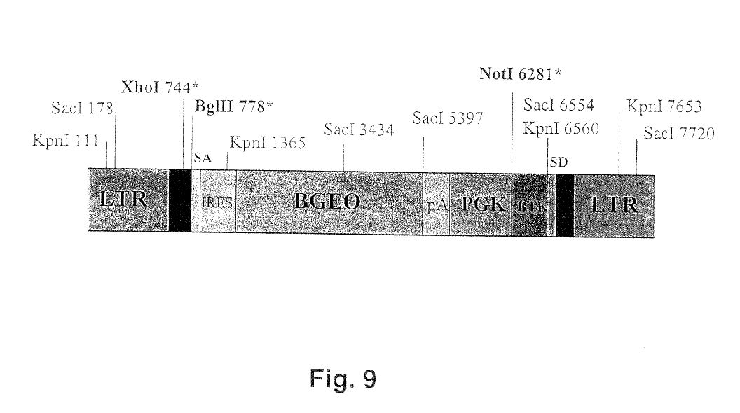

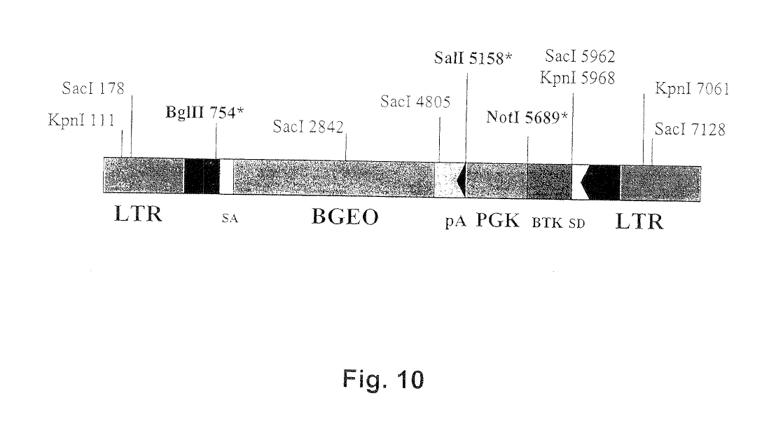

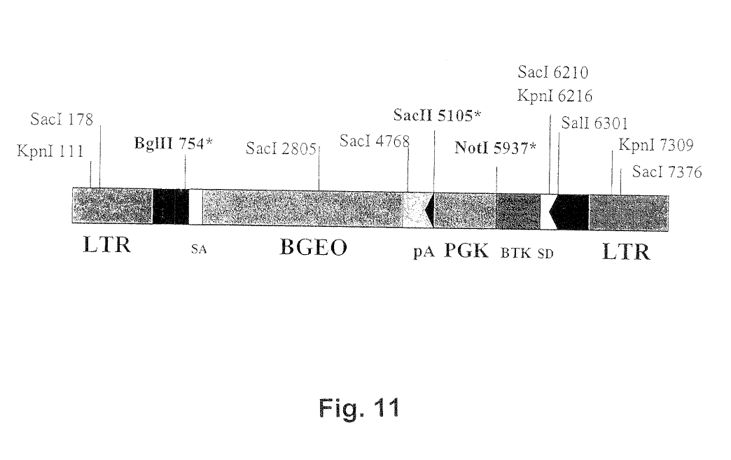

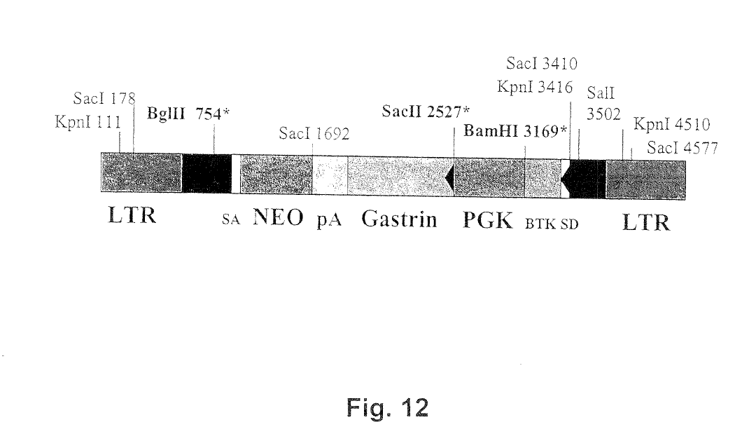

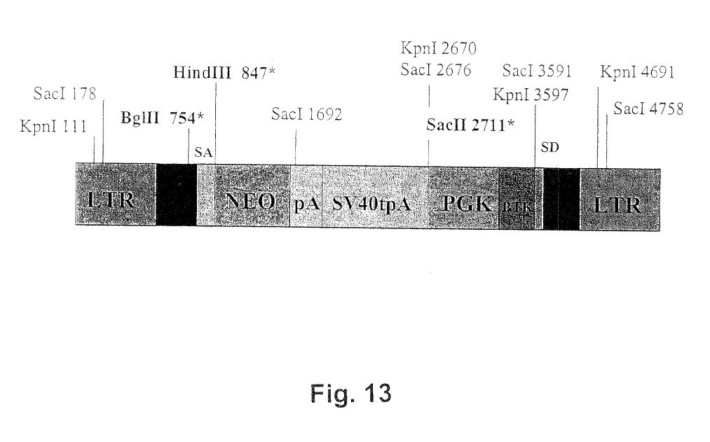

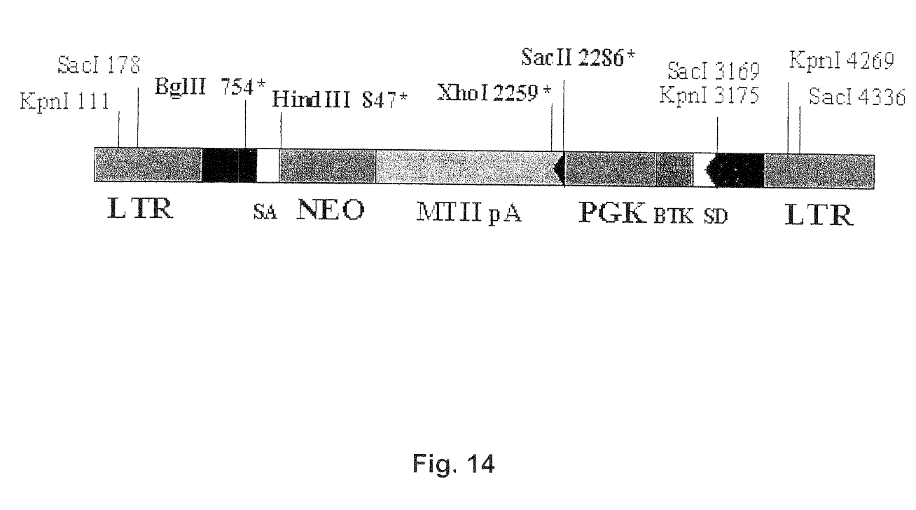

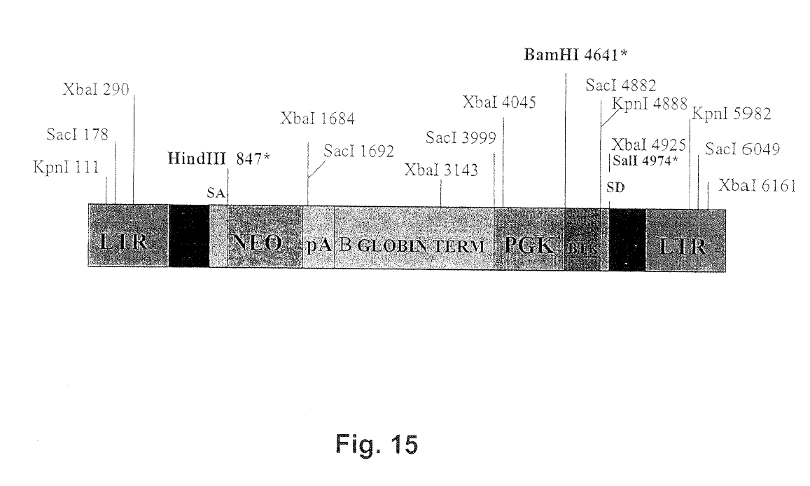

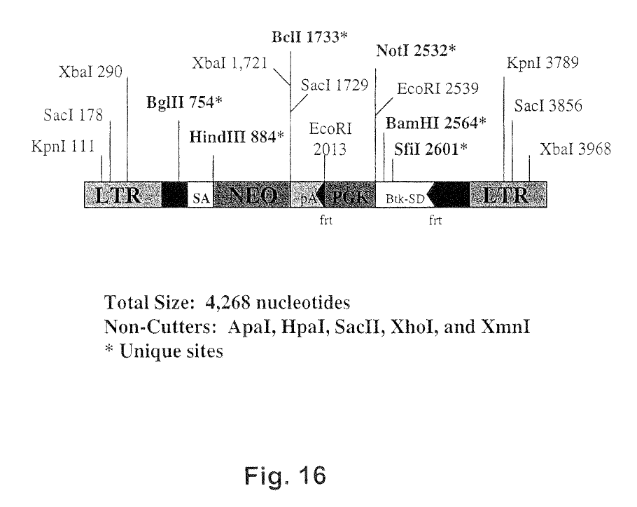

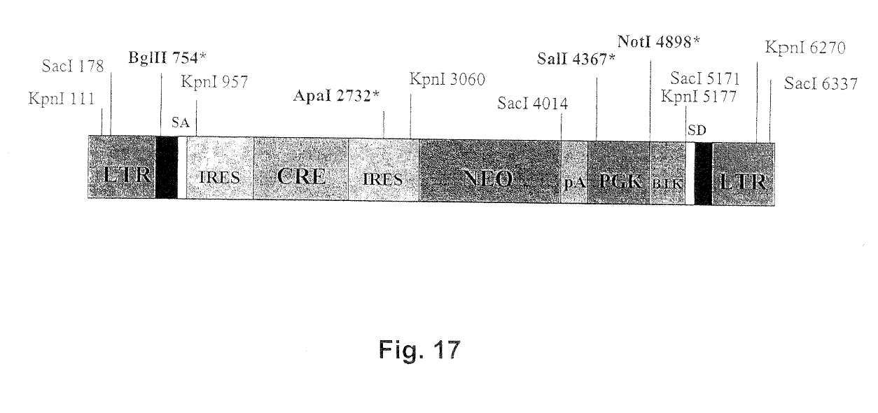

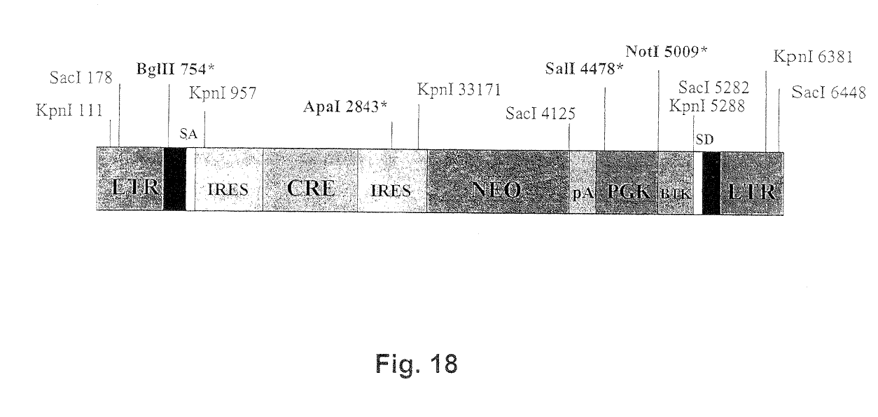

In certain embodiments, a genomic mutation of the current invention is engineered by introducing a vector as shown in one or more of FIGS. 1 through 18 into a mouse cell, for example, an embryonic stem cell. A vector as shown in each of FIGS. 1 through 18 is referred to as a gene trap vector. In certain preferred embodiments, a gene trap vector may integrate into a gene in the genome of a cell. In certain other preferred embodiments, the integration of a gene trap vector into a gene results in a transcript from that gene comprising vector sequences and gene sequences (hybrid transcript). In certain other preferred embodiments, a hybrid transcript results from a gene with an integrated gene trap vector after transcription of the gene and before and following splicing of the transcript. Elements of gene trap vectors useful for making a mouse or cell of the current invention are known to those of skill in the art, for example, a mouse PGK promoter is discussed in Adra et al., 1987, Gene 60(1):65-74; a mouse BTK sequence is discussed in Sideras et al., 1994, J. Immunol. 153(12):5607-17; an SABgeo sequence and a splice acceptor sequence from adenovirus are discussed in Friedrich and Soriano, 1991, Genes Dev. 5(9):1513-23; a pGen (MoMuLV backbone) sequence is discussed in Soriano et al., 1991, J. Virol. 65(5):2314-9; a bGH polyadenylation sequence and a SV40 polyadenylation sequence are discussed in Pfarr et al., 1986, DNA 5(2):115-22, a polyadenylation signal from the major viral coat protein 1 gene of the SV40 virus is discussed in Maxwell et al, 1989, Biotechniques. 7(3):276-80, all of which are incorporated by reference.

In certain other preferred embodiments, a mutation is engineered into a cell that is maintained in culture or any environment that allows the manipulation of large numbers of cells and that allows the screening and identification of cells with desired characteristics, for example, cells that comprise an engineered genomic mutation of interest. In certain other embodiments, cells are maintained, grown, manipulated, screened and/or identified using one, two, three, four, or more different cultures, media, environments, buffers, sera, salts, and supports. See, for example, U.S. Pat. Nos. 6,875,607; 6,878,542; 6,815,185 for background on tissue culture, all of which are incorporated herein by reference for all purposes.

In certain other embodiments, a genomic mutation is engineered into a mouse by introducing said mutation into the cells of a mouse. For example, an oligonucleotide or a polynucleotide may be introduced into the cells of a mouse by any means capable of such delivery, for example, a virus, a liposome or any other means known in the art.

Background on generating a mouse library, including cloning, tissue culture, mutagenesis and generating mice, is also described in U.S. Patent Application Nos. 20020182724, 20040072243, 20040259253, 20050059060, 20050053953; and in U.S. Pat. Nos. 6,080,576; 6,136,566; 6,207,371; 6,218,123; 6,436,707; 6,776,988; 6,808,921; 6,855,545, all of which are incorporated herein by reference for all purposes.

### 6.1.2 Characterizing Mice of the Libraries of the Current Invention

Mice of a library of the current invention, in certain embodiments, are characterized by analyzing mouse lines comprising said library. A mouse line, in certain embodiments, consists of mice that are related as off-spring. In certain other embodiments, a mouse line consists of mice that comprise the same engineered genomic mutation. A mouse line, in certain embodiments, comprises mice that are homozygous or heterozygous for the same engineered genomic mutation, mice that are chimeras comprising cells that comprise that same engineered genomic mutation, and mice that are off-spring (or progeny) of such homozygotes, heterozygotes or chimeras, including wild-type off-spring that does not carry the same engineered genomic mutation. A mouse of each of these aforementioned types that is part of a mouse line of the invention is referred to herein as Homozygote, Heterozygote, Chimera, Off-Spring or Wild-Type. In certain embodiments, at least 50 percent of all mouse lines comprising a library of the invention are characterized, in certain other preferred embodiments, at least 75 percent or all mouse lines of such a library are characterized.

A mouse line of the present invention, in certain embodiments, is characterized by analyzing a Homozygote, a Heterozygote, a Chimera, an Off-Spring, and a Wild-Type of said mouse line, or any one, two, three or four thereof. In certain embodiments, at least one Homozygote, one Heterozygote and one Wild-Type mouse is analyzed, or at least two of each of the three types, at least three, at least four, at least five, at least eight, at least ten, or at least twenty. A mouse is characterized, in certain embodiments, by analyzing any aspect of the mouse or relating to the mouse. In certain embodiments, a mouse is characterized by analyzing all or a part of its structure, function, anatomy, physiology, genetics, genome, gene expression, cell growth, tumorigenesis, development, embryology, behavior, movement, susceptibility to disease, life span, weight, skeleton, or any other aspect of the mouse. A Homozygote or Heterozygote mouse of the current invention is characterized by at least identifying a gene affected by an engineered genomic mutation of said mouse.

### 6.1.2.1 Genetic Analysis

A mouse of the invention, in certain embodiments, is analyzed genetically. A preferred genetic analysis comprises determining or identifying any change or changes in the genome of a mouse comprising an engineered genomic mutation when compared to a mouse that does not comprise such a mutation. A genetic analysis, according to certain embodiments, comprises identifying a part of the genome affected by the engineered mutation, isolating a part of the genome comprising the engineered mutation, cloning a part of the genome comprising the engineered mutation, sequencing DNA, sequencing RNA, performing a restriction analysis, determining the site where an exogenous polynucleotide integrated into the genome, determining the presence of a rearrangement of parts of the genome, determining if a part of the genome has been deleted, determining if a part of the genome has been multiplied, determining if a part of the genome has been modified, or determining if any other kind of change has occurred to the genome of a mouse of the invention. See, for example, U.S. Pat. Nos. 6,875,607; 6,867,035; 6,878,542; 6,815,185; 6,884,622 for background on cloning.

Background on genetic analysis is also described in U.S. Patent Application Nos. 20020182724, 20040072243, 20040259253, 20050059060, 20050053953; and in U.S. Pat. Nos. 6,080,576; 6,136,566; 6,207,371; 6,218,123; 6,436,707; 6,776,988; 6,808,921; 6,855,545, all of which are incorporated herein by reference for all purposes.

### 6.1.2.2 Transcriptional Analysis

A mouse of the invention, in certain embodiments, is analyzed by performing a transcriptional analysis. A transcriptional analysis, according to certain embodiments, comprises determining the activity of any part of the genome of a mouse, for example, the activity of one or more genes. A transcriptional analysis, according to certain embodiments, comprises determining the quantity, stability, structure, location, sequence, modification, binding, or folding of RNA, for example, mRNA, hnRNA, tRNA, rRNA, or any other kind of RNA.

A transcriptional analysis comprises, in certain preferred embodiments, identifying an RNA molecule found in a mouse carrying the engineered genomic mutation but not in a mouse of the same strain not carrying that mutation (“mutated RNA”). In certain embodiments, a mutated RNA results from transcription of a part of the genome altered by the engineered genomic mutation, for example, if the engineered mutation resulted in the insertion of an exogenous sequence into an exon of a gene, transcription of that gene would result in an RNA molecule that includes the exogenous sequence. Or, for example, where the engineered mutation inserts exogenous exon sequences and one or more splice sites into a gene (for example, an exogenous exon with a splice acceptor site upstream and a splice donor site downstream may integrate into an intron) transcription of that gene would result in an RNA molecule that includes the exogenous exon sequences and the splice sites (“mutated hnRNA”). A mutated hnRNA, in certain embodiments, would undergo splicing to yield mature RNA molecules found in a mouse with an engineered genomic mutation (“mutated mRNA”) but not in a Wild-Type mouse (for example, the mutated mRNA would include an exogenous exon sequence that is not found in mRNA resulting from the non-mutated gene).

Background on transcriptional analysis is also described in U.S. Patent Application Nos. 20020182724, 20040072243, 20040259253, 20050059060, 20050053953; and in U.S. Pat. Nos. 6,080,576; 6,136,566; 6,207,371; 6,218,123; 6,436,707; 6,776,988; 6,808,921; 6,855,545, all of which are incorporated herein by reference for all purposes.

### 6.1.2.3 Phenotypic Analysis

A mouse of the invention, in certain embodiments, is analyzed by performing a phenotypic analysis. A phenotypic analysis, according to certain embodiments, comprises an assessment or evaluation of the behavior, coat color, size, proportions, shape, skeleton, reflexes, blood, blood protein, blood composition, cholesterol level, life span, weight, organ size, organ shape, organ function, brain size, or any other phenotypic aspect.

### 6.1.2.4 Computational Analysis

A mouse of the invention, in certain embodiments, is analyzed by performing a computational analysis. A computational analysis, according to certain embodiments, comprises a comparison of data derived from a mouse, a mouse line or a mouse library of the current invention with data known in the art. In certain other embodiments, a computational analysis comprises a comparison of data derived from a mouse or a mouse line of the current invention with data obtained from another mouse or mouse line of the current invention.

A computational analysis, in certain embodiments, comprises a comparison of genomic data, sequence data, expression data, phenotypic data, behavioral data, and any other kind of data.

### 6.2 Mice of the Current Invention

The current invention provides, in certain embodiments, a mouse carrying an engineered genomic mutation and, in certain other embodiments, the current invention provides a mouse line of the mice of the current invention. A mouse of the current invention, in certain embodiments, may be a Homozygote, a Heterozygote, a Chimera, an Off-Spring or a Wild-Type.

A mouse of the current invention may be maintained, kept or stored, in certain embodiments, as a living animal, an ES cell, an egg, a fertilized egg, a gamete, a morula, a blastocyst, an embryo, an oocyte, a fetus, or a cell of any cell type or cell aggregate that is capable of generating a mouse. A mouse of the current invention may be stored, in certain embodiments, through cryopreservation. In certain embodiments, cryopreservation is not used to store a living animal. Methods for cryopreservation useful to store a mouse of the current invention are known in the art and are described, for example, in U.S. Pat. Nos. 5,962,213; 6,361,934; 6,500,608; 6,503,698; 6,519,954; and in Van den Abbeel et al, 1994, Cryobiology 31(5):423-33; Dumoulin etaL, 1994, Fertil Steril. 62(4):793-8; Vasuthevan et al., 1992, J. Assist. Reprod. Genet. 9(6):545-50; Thornton et al., 1999, Mamm. Genome 10(10):987-92; Songsasen and Leibo, 1997, Cryobiology 35(3):255-69; Songsasen and Leibo, 1997, Cryobiology 35(3):240-54; Songsasen et al., 1997, Biol. Reprod. 56(1):143-52; Critser and Mobraaten, 2000, ILAR J. 41(4):197-206; Kasai and Mukaida, 2004, Reprod. Biomed. Online 9(2):164-170; Menezo, 2004, Obstet. Gynecol. Reprod. Biol. 115 Suppl 1:S12-5; Sztein et al., 2001, Cryobiology 42(1):28-39; Sztein et al., 1999, Lab.-Anim.-Sci. 49(1):99-100; Sztein et al., 1998, Biol.-Reprod. 58(4): 1071-4, all of which are incorporated herein by reference for all purposes.

Table 1 shows examples of mice carrying engineered genomic mutations of the current invention. Table 1 also includes a description of the phenotype of a mouse carrying each of the engineered genomic mutations in Table 1.

Background on mice is also described in U.S. Patent Application Nos. 20020182724, 20040072243, 20040259253, 20050059060, 20050053953; and in U.S. Pat. Nos. 6,080,576; 6,136,566; 6,207,371; 6,218,123; 6,436,707; 6,776,988; 6,808,921; 6,855,545, all of which are incorporated herein by reference for all purposes.

### 6.3 Cells of the Current Invention

The current invention further includes, in certain embodiments, cells carrying an engineered genomic mutation. A cell of the current invention includes, in certain embodiments, a stem cell, an embryonic stem cell, a hematopoietic stem cell, a progenitor cell, a fibroblast, a nerve cell, a muscle cell, an epithelial cell, a teratocarcinoma cell, any other cell type known in the art, a cell that is progeny of one of the mentioned cell types, a cell that is derived from one of the mentioned cell types through differentiation.

A cell of the current invention may be obtained, in certain embodiments, by engineering a mutation into the genome of such a cell, for example, a stem cell or an embryonic stem cell. A cell of the current invention may also be obtained, in certain embodiments, from a mouse of the current invention, for example, from a Homozygote, a Heterozygote, an Off-Spring, a Chimera, or a Wild-Type. In certain other embodiments, a cell of the current invention is progeny of a cell obtained from a mouse of the current invention, for example, from a Homozygote, a Heterozygote, an Off-Spring, a Chimera, or a Wild-Type.

Background on cells is also described in U.S. Patent Application Nos. 20020182724, 20040072243, 20040259253, 20050059060, 20050053953; and in U.S. Pat. Nos. 6,080,576; 6,136,566; 6,207,371; 6,218,123; 6,436,707; 6,776,988; 6,808,921; 6,855,545, all of which are incorporated herein by reference for all purposes.

### 6.4 Polynucleotides of the Current Invention

The current invention provides, in certain embodiments, a polynucleotide. In certain embodiments, a polynucleotide of the current invention is derived from a cell or a mouse of the current invention. In certain other embodiments, a polynucleotide of the current invention comprises one, more than one, or all exons and/or one, more than one, or all introns of a gene that is found in a Wild-Type of a mouse line of the current invention and that carries an engineered genomic mutation in a Homozygote of the same mouse line, or its complementary, or a fragment thereof, or a polynucleotide capable of hybridizing thereto under conditions of moderate stringency or high stringency. A polynucleotide of the current invention, in certain embodiments, encodes a polypeptide of the current invention. A polynucleotide of the current invention may be DNA, RNA or any modification or derivative thereof. A polynucleotide may be genomic DNA, cDNA, hnRNA, mRNA, or any combination thereof. A polynucleotide of the current invention is at least 50 base pairs in length, or at least 100 base pairs, or at least 200 base pairs, or at least 300 base pairs, or at least 400 base pairs, or at least 500 base pairs, or at least 1000 base pairs, or at least 2000 base pairs, or at least 5000 base pairs, or at least 10000 base pairs, or at least 20000 base pairs in length.

The identification, isolation, cloning, modification and other processing of polynucleotides and conditions for hybridization under various conditions, including moderate and high stringency, are described in United States Patent Application Number 20050059060, which is incorporated herein for that and any other purpose.

### 6.5 Polypeptides of the Current Invention

The current invention provides, in certain embodiments, a polypeptide. In certain embodiments, a polypeptide of the current invention is derived from a cell or a mouse of the current invention. In certain other embodiments, a polypeptide of the current invention comprises a polypeptide that is encoded by one, more than one, or all exons of a gene that is found in a Wild-Type of a mouse line of the current invention and that carries an engineered genomic mutation in a Homozygote of the same mouse line, or a fragment thereof. In certain other embodiments, a polypeptide of the current invention is encoded by a polynucleotide of the current invention. A polypeptide of the current invention may comprise any one or more or all of the naturally occurring amino acids, or any modification or derivative thereof. A polypeptide of the current invention is at least 10 amino acids in length, or at least 25 amino acids, or at least 50 amino acids, or at least 100 amino acids, or at least 200 amino acids, or at least 300 amino acids, or at least 500 amino acids in length.

The identification, isolation, modification and other processing of polypeptides are described in United States Patent Application Number 20050059060, which is incorporated herein for that and any other purpose.

### 6.6 Antibodies of the Current Invention

The current invention also provides antibodies. In certain embodiments, an antibody of the present invention is specific to an antigen or epitope which is absent in a mouse of the invention (Absent Antigen). In certain preferred embodiments, an Absent Antigen is absent in a Homozygote of a mouse line of the invention, but not absent in a Wild-Type of the same mouse line. In certain other embodiments, an Absent Antigen is synthesized in a cell of a Wild-Type of a mouse line of the invention, but not in a Homozygote of the same mouse line. In certain embodiments, an Absent Antigen is expressed by a gene in a Wild-Type of a mouse line of the invention but not a Homozygote of the same mouse line. In certain other embodiments, an Absent Antigen is encoded by a gene in a Wild-Type of a mouse line of the invention but not a Homozygote of the same mouse line.

In certain other embodiments, an antibody of the invention is raised in a mouse of the invention, for example, a Wild-Type, a Chimera, an Off-Spring, a Heterozygote, and preferably a Homozygote. In certain other embodiments, an antibody of the current invention may be changed or altered into another antibody type. For example, a mouse antibody may be altered into a humanized antibody with the same antigen specificity, or any other type of alteration may be carried out. Antibody types contemplated under the current invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. In certain embodiments, an antibody of the current invention binds an Absent Antigen with a binding affinity (Kₐ) of 10⁶ M⁻¹ or greater, preferably 10⁷ M⁻¹ or greater, more preferably 10⁸ M⁻¹ or greater, more preferably 10⁹ M⁻¹ or greater, more preferably 1010 M⁻¹ or greater, more preferably 10¹¹ M⁻¹ or greater, and most preferably 10¹² M⁻¹ or greater.

Background on antibodies, antibody generation, characterization, modification, and uses are also described in U.S. Patent Application Nos. 20020182724, 20040072243, 20040259253, 20050059060, 20050053953; and in U.S. Pat. Nos. 6,080,576; 6,136,566; 6,207,371; 6,218,123; 6,436,707; 6,776,988; 6,808,921; 6,855,545; 6,824,780; 6,827,934; 6,824,993; 6,767,541; 6,828,425, all of which are incorporated herein by reference for all purposes.

### 6.7 Therapeutic Utility

The present invention provides, in certain embodiments, efficient drug development or development of therapies, or both. A mouse or a cell of the present invention may be used to obtain drugs and therapies for treatment of disease. An antibody of the present invention, in certain embodiments, may be used for therapeutic applications. Therapeutic applications, for example of antibodies of the invention, include disease phenotypes and phenotypic traits as those shown in Table 1.

### 6.8 Diagnostic Utility

The present invention provides, in certain embodiments, diagnostic tools. For example, a mouse or a cell of the present invention may be used, in certain embodiments, to diagnose the effectiveness of drugs, antibodies, or small molecules. An antibody of the present invention may be used, for example, to diagnose the presence or quantity of an antigen or epitope that the antibody is specific for, or a disease condition associated with a phenotype found in the animal in which the antibody is raised.

The present invention is further illustrated by the following examples, which are not intended to be limiting in any way whatsoever.

### EXAMPLES

### Example 1

### Generation of a Library of Mutated Mouse ES Cells Defined By GTS Sequences

Retroviral vectors such as those exemplified and described in detail in U.S. Pat. Nos. 6,080,576, 6,136,566, 6,139,833 were used to generate a collection of gene trapped ES cell clones. Plasmids containing various VICTR cassettes described above were constructed by conventional cloning techniques. Usually, the cassettes contained a PGK promoter directing transcription of an exon that ends in a canonical splice donor sequence. The transcript encoding the exon was engineered to contain sequences that allow for the annealing of two nested PCR and sequencing primers. The vector backbone was based on pBluescript KS+from Stratagene Corporation.

The plasmid construct was linearized by digestion with, for example, ScaI, which cuts at a unique site in the plasmid backbone. The plasmid was then transfected into the mouse ES cell line AB2.2 by electroporation using a BioRad Genepulser apparatus. After the cells were allowed to recover, gene trap clones were selected by adding puromycin to the medium at a final concentration of 3 μg/ml (other antibiotics, such as G418, were used at suitable concentrations as applicable). Positive clones were allowed to grow under selection for approximately 10 days before being removed and cultured separately for storage and to determine the sequence of the disrupted gene.

Total RNA was isolated from an aliquot of cells from each of 18 gene trap clones chosen for study. Five micrograms of this RNA was used in a first strand cDNA synthesis reaction using the “RS” primer. This primer has unique sequences (for subsequent PCR) on its 5′ end and nine random nucleotides or nine T (thymidine) residues on it's 3′ end. Reaction products from the first strand synthesis were added directly to a PCR with outer primers specific for the engineered sequences of puromycin and the “RS” primer. After amplification, aliquots of reaction products were subjected to a second round of amplification using primers internal, or nested, relative to the first set of PCR primers. This second amplification provided more reaction product for sequencing and also provided increased specificity for the specifically gene trapped DNA.

The products of the nested PCR were visualized by agarose gel electrophoresis, and seventeen of the eighteen clones provided at least one band that was visible on the gel with ethidium bromide staining. Most gave only a single band, which is an advantage in that a single band is generally easier to sequence. The PCR products were sequenced directly after excess PCR primers and nucleotides were removed by filtration in a spin column (Centricon-100, Amicon). DNA was added directly to dye terminator sequencing reactions (purchased from ABI) using the standard M13 forward primer, a region for which was built into the end of the puro exon in all of the PCR fragments.

Subsequent studies have used both VICTR 3, VICTR 20 and follow-on vectors. Like VICTR 3, VICTR 20 is exemplary of a broader family of vectors that incorporate two main functional units: a sequence acquisition component having a strong promoter element (phosphoglycerate kinase 1) active in ES cells that is fused to the puromycin resistance gene coding sequence that lacks a polyadenylation sequence but is followed by a synthetic consensus splice donor sequence (PGKpuroSD); and 2) a mutagenic component that incorporates a splice acceptor sequence fused to a selectable, calorimetric marker gene and followed by a polyadenylation sequence (for example, SAβgeopA or SAIRESβgeopA). Also like VICTR 3, stop codons have been engineered into all three reading frames in the region between the 3′ end of the selectable marker and the splice donor site.

When VICTRs 3, 20, and various variations and modifications thereof were used in the commercial scale application of the presently disclosed invention; many mutagenized ES cell clones were rapidly engineered and obtained. Sequence analysis obtained from these clones has identified a wide variety of both previously identified and novel sequences.

The cloned 3′ RACE products resulting after the target ES cells were infected with VICTR 20 were purified using conventional column chromatography (e.g., S300 and G-50 columns), and the products were recovered by centrifugation. Purified PCR products were quantified by fluorescence using PicoGreen (Molecular Probes, Inc., Eugene Oreg.) as per the manufacturer's instructions.

Dye terminator cycle sequencing reactions with AmpliTaq® FS DNA polymerase (Perkin Elmer Applied Biosystems, Foster City, Calif.) were carried out using approximately 7 pmoles of sequencing primer, and approximately 30-120 ng of 3′ template. Unincorporated dye terminators were removed from the completed sequencing reactions using G-50 columns as described above. The reactions were dried under vacuum, resuspended in loading buffer, and electrophoresed through a 6% Long Ranger acrylamide gel (FMC BioProducts, Rockland, Me.) on an ABI Prism® 377 with XL upgrade as per the manufacturer's instructions. The sequences of the resulting amplicons, or GTSs, were recorded.

### Example 2

### Transfecting ES Cells

a. Vector Construction

The promoter from the mouse phosphoglycerate kinase (PGK) gene was placed upstream from the first exon of the naturally occurring murine btk gene (nucleotides 40,043 to 40,250 of the murine btk gene). The first exon of the btk gene does not contain a translational start site and initiation codon marking the 5′ region of the coding sequence; however, these features could be engineered into the exon if desired. The 3′ end of the coding region of the first exon is marked by a splice donor sequence. Given that splice donor recognition sequences can extend into intronic sequence, 103 bases of intron DNA was retained after the end of the btk first exon. The PGKbtkSD cassette lacks a 3′ polyadenylation signal. Accordingly, any transcript produced by the cassette cannot be properly processed, and therefore identified by 3′ RACE, unless the transcript is spliced to a 3′ exon that can be polyadenylated.

The above 3′ gene trap cassette was placed into a retroviral vector (in reverse orientation relative to the flanking LTR regions) that incorporated a polyadenylation site 5′ to the PGK promoter of the 3′ gene trap cassette, the neo gene was placed 5′ to the polyadenylation site, and a splice acceptor (SA) site was placed 5′ to the neo coding region to produce a functional SAneopA, or optionally a SAIRESneopA 5′ gene trap cassette. This vector also incorporates, in operable combination, a pair of recombinase recognition sites that flank the PGKbtkSD cassette. This vector typically requires that the target cell naturally express the trapped gene; however, this requirement can be overcome by adding a promoter that independently controls the expression of the selectable marker.

b. 3′ Gene Trapping

The btk vector was introduced into the embryonic stem cells using standard techniques. In brief, supernatant from GP+E packaging cells was added to approximately 2×10⁶ embryonic stem cells (at an input ratio of approximately 0.1 virus/target cell) for 16 hours and the cells were subsequently selected with G418 for 10 days. G418 resistant cells were subsequently isolated, grown up on 96-well plates and subjected to automated RNA isolation, reverse transcription, PCR and sequencing protocols to obtain the gene trapped sequences.

RNA Isolation was carried out on DNA bind plates (Corning/Costar) treated with 5′-amino (dT)₄₂ (GenoSys Biotechnologies) in a 50 mM Sodium Phosphate buffer, pH 8.6, and allowed to sit at room temperature overnight. Immediately prior to use the plates were rinsed three times with PBS and twice with TE. Cells were rinsed with PBS, lysed with a solution containing 100 mM Tris-HCl, 500 mM LiCl, 10 mM EDTA, 1% LiDS, and 5 mM DTT in DEPC water, and transferred to the DNA binding plate where the mRNA was captured. After a 15 minute incubation the RNA was washed twice with a solution containing 10 mM Tris-HCl, 150 mM LiCl, 1 mM EDTA, and 0.1% LiDS in DEPC water. The RNA was then rinsed three times with the same solution minus LiDS. Elution buffer containing 2 mM EDTA in DEPC water was added and the plate was heated at 70° C. for five minutes. An RT premix containing 2× First Strand buffer, 100 mM Tris-HCl, pH 8.3, 150 mM KCl, 6 mM MgCl₂, 2 mM dNTPs, RNAGuard (1.5 units/reaction, Pharmacia), 20 mM DTT, QT primer (3 pmol/rxn, GenoSys Biotechnologies, sequence: 5′ CCAGTGAGCAGAGTGACGAGGACTCGAGCTCAAGCTTTTTTTTTTTTTTTTT 3′, SEQ ID NO: 1) and Superscript II enzyme (200 units/r×n, Life Technologies) was added. The plate was transferred to a thermal cycler for the RT reaction (37° C. for 5 min. 42° C. for 30 min. and 55° C. for 10 min).

### Example 3

### Genetic Analysis of ES Cell Colonies

The cDNA was amplified using two rounds of PCR. The PCR premix contains: 1.1×MGBII buffer (74 mM Tris pH 8.8, 18.3 mM Ammonium Sulfate, 7.4 mM MgCl₂, 5.5 mM 2ME, 0.011% Gelatin), 11.1% DMSO (Sigma), 1.67 mM dNTPS, Taq (5 units/r×n), water and primers. The sequences of the first round primers are: Pₒ 5′AAGCCCGGTGCCTGACTAGCTAG3′, SEQ ID NO: 2; BTK, 5′GAATATGTCTCCAGGTCCAGAG3′, SEQ ID NO: 3; and Qₒ 5′ CCAGTGAGCAGAGTGACGAGGAC3′, SEQ ID NO: 4 (pmol/r×n). The sequences of the second round primers are Pᵢ 5′CTAGCTAGGGAGCTCGTC3′, SEQ ID NO: 5; BTKᵢ 5′ CCAGAGTCTTCAGAGATCAAGTC3′, SEQ ID NO: 6; and Qᵢ 5′ GAGGACTCGAGCTCAAGC3′, SEQ ID NO: 7 (50 μmol/r×n). The outer premix was added to an aliquot of cDNA and run for 17 cycles (95° C. for 1 min. 94° C. for 30 sec., 58° C. for 30 sec 65° C. for 3.5 min). An aliquot of this product was added to the inner premix and cycled at the same temperatures 40 times.

The nested 3′ RACE products were purified in a 96-well microtiter plate format using a two-step protocol as follows. Twenty-five microliters of each PCR product was applied to a 0.25 ml bed of Sephacryl®. S-300 (Pharmacia Biotech AB, Uppsala, Sweden) that was previously equilibrated with STE buffer (150 mM NaCl, 10 mM Tris-HCL, 1 mM EDTA, pH 8.0). The products were recovered by centrifugation at 1200×g for 5 minutes. This step removes unincorporated nucleotides, oligonucleotides, and primer-dimers. Next, the products were applied to a 0.25 ml bed of Sephadex® G-50 (DNA Grade, Pharmacia Biotech AB) that was equilibrated in MilliQ H₂O, and recovered by centrifugation as described earlier. Purified PCR products were quantified by fluorescence using PicoGreen (Molecular Probes, Inc., Eugene Oreg.) as per the manufacturer's instructions.

Dye terminator cycle sequencing reaction with AmpliTaq® FS DNA polymerase (Perkin Elmer Applied Biosystems, Foster City, Calif.) were carried out using 7 pmoles of primer (Oligonucleotide OBS; 5′CTGTAAAACGACGGCCAGTC3′, SEQ ID NO: 8) and approximately 30-120 ng of 3′ RACE product. The cycling profile was 35 cycles of 95° C. for 10 sec, 55° C. for 30 sec, and 60° C. for 2 min. Unincorporated dye terminators were removed from the completed sequencing reactions using G-50 columns as described earlier. The reactions were dried under vacuum, resuspending in loading buffer, and electrophoresed through a 6% Long Ranger acrylamide gel (FMC BioProducts, Rockland, Me.) on an ABI Prism® 377 with XL upgrade as per the manufacturer's instructions.

The automated 96-well format was used to obtain sequence, and data was obtained from 70% of the colonies. Upon examination, the sequence from the first exon of btk was identified followed by the btk splice junction. The splice junction was followed by unique sequences from each separate gene trap event. These sequences averaged 500 bp in length and were of high quality often containing long open reading frames. In addition 80% of these sequences can be matched using blast searches to sequences found in the GenBank database indicating that transcribed exonic sequences were identified. These gene trap sequence tags are of significantly better length and quality than those produced by previous gene trap designs. The new tags are improved in both length and quality and the fact that 80% of the tags match GenBank sequences suggests that they efficiently trap genes.

### Example 4

### Using ES Cells to Generate Mice

ES cells of the current invention are used to generate a mouse of the current invention. Any technique known in the art can be employed to generate a mouse with an ES cell. For example, an ES cell can be used to generate a mouse by injecting the cell into a blastocyst as described, for example, in Bradley, 1987. In: Teratocarcinomas and Embryonic Stem Cells: A Practical Approach, Robertson, ed. IRL, Oxford, pp. 113-152. Blastocysts can be isolated from a pregnant mouse on day 3.5 of pregnancy. About 20-25 ES cells are typically injected into a blastocyst. Following injection, the blastocyst is implanted into a suitable pseudopregnant female foster animal and the embryo brought to term. Progeny of the foster animal are used to breed more animals. If an ES cell harboring the engineered genomic mutation entered the germline of the foster animal's Off-Spring, the mutation will be represented in the Off-Spring's gene pool. Animals are bred to obtain a Homozygote, Heterozygote, Chimera, Off-Spring and/or Wild-Type animal. One may determine if an ES cell carrying an engineered genomic mutation of interest has entered the germline by observing the coat color of Off-Spring mice. As the ES cells are derived from an agouti coat color strain and the blastocyst from a black coat color strain, second or higher generation Off-Spring carrying agouti coat color would be indicative that an ES cell carrying the engineered genomic mutation has entered the germline. In addition, Off-Spring animals are examined genetically to detect the presence of the genetrap vector and to characterize the engineered genomic mutation.

Additional background on generating a mouse can also be found in, for example, Puhler, A., Ed., Genetic Engineering of Animals, VCH Publ., 1993; Murphy and Carter, Eds., Transgenesis Techniques Principles and Protocols (Methods in Molecular Biology, Vol. 18), 1993; and Pinkert, C A, Ed., Transgenic Animal Technology: A Laboratory Handbook, Academic Press, 1994. Transgenic mice may also be generated, for example, as described in Thomas et al. (1999) Immunol., 163:978-84; Kanakaraj et al. (1998) J. Exp. Med., 187:2073-9; or Yeh et al. (1997) Immunity 7:715-725; Jaenisch (1988) Science, 240:1468-1474.

### Example 5

### Phenotypic Analysis of Mice

Functional annotation of the mammalian genome in the post-genome era is an important task. Genetic studies in model organisms provide an approach for understanding gene function. Technologies for parallel production and analysis of mouse mutants provides a valuable screen through mutations in druggable genes to identify targets for drug discovery. By carrying out genetic screens in a mammalian model system, one can screen directly for changes in physiology relevant to disease treatment. This example describes a biological screen for analyzing large numbers of mouse lines, for example, for screening 1000 mouse gene knockouts per year. This screen is focused on discovering the targets for therapeutic products in the areas of metabolism, endocrinology, immunology, neurology, cardiology, opthalmology, reproductive biology and oncology.

### 5.1 INTRODUCTION

Genetic screens can be used to scan a genome for genes that play a role in any process of interest. Genetic screens were carried out in invertebrate model organisms and included saturation screens of Drosophila Model Organisms in Drug Discovery. Ed. Pamela M. Carroll and Kevin Fitzgerald Copyright 2003 John Wiley & Sons, Ltd., to identify genes involved in organization of the body plan during development (Nusslein-Volhard and Wieschaus, 1980) and screens in Caenorhabditis elegans to identify genes involved in producing the invariant cell lineage pattern (Horvitz and Sulston, 1980; Chalfie et al., 1981; Hedgecock et al., 1983). These screens relied on saturation mutagenesis to interrogate the genome for the set of genes involved in these processes and led to the discovery of genes such as the homeobox genes and apoptosis regulators involved in development across all invertebrate and vertebrate species examined. Since these screens, additional genetic screens have been carried out in the fly and worm, further demonstrating the power of genetics for the dissection of pathways and processes. These screens require only a method for creating large numbers of tractable mutations in genes and a phenotype that can be measured.

Genetic screening has been adapted for the vertebrate model organisms of zebrafish and mice. In zebrafish, both chemical mutagenesis (Mullins et al., 1994; Haffter et al., 1996) and gene trapping (Golling et al., 2002) have been combined with phenotypic screens to identify mutations affecting development of the neural crest, pigmentation, jaw, branchial arches, visual system, heart and other internal organs, ear, retina, brain, midline, shape and movement (Brockerhoff et al., 1995; Abdelilah et al., 1996; Baier et al., 1996; Brand et al., 1996; Chen et al., 1996; Granato et al., 1996; Kelsh et al., 1996; Malicki et al., 1996a,b; Neuhauss et al., 1996; Odenthal et al., 1996; Piotrowski et al., 1996; Schier et al., 1996; Solnica-Krezel et al., 1996; Stemple et al., 1996). These screens take advantage of the large number of Off-Spring, oviparous development and transparent nature of the zebrafish embryo that make it a system for the study of vertebrate development that should result in the identification of genes involved in vertebrate development. For the purpose of drug discovery, effective genetic screens in mammals allow one to dissect mammalian physiology to identify key genes with therapeutic relevance as potential drug targets. Genetic screens in mammals are logistically possible with advances in mutagenesis and screening methods in mice that facilitate functional dissection of the mammalian genome. Advances in the scale and speed of gene targeting (Walke et al., 2001; Abuin et al., 2002) and the development of genome-wide gene trapping (Zambrowicz et al., 1998; Wiles et al., 2000; Leighton et al., 2001; Mitchell et al., 2001) in mouse embryonic stem cells resulted in mutations in a number of genes. The screen described in this example combines mouse screening methods with miniaturization of medical technologies and with disease challenge assays useful to the mouse model for diagnostic analysis of mice. The mouse is a model organism that is ideal for studying many aspects of mammalian physiology with direct medical relevance. Screens are used to identify genes involved in insulin sensitivity, hypertension, body fat deposition, energy expenditure, bone deposition and breakdown, angiogenesis and many other processes with significance for the treatment of disease.

These advances have brought together two aspects for genetic screens in mammals: the ability to produce large numbers of mutations and the ability to screen for phenotypes of interest. The development of mutagenesis strategies to mutate large numbers of mouse genes has been described (Zambrowicz et al., 1998; Wiles et al., 2000; Leighton et al., 2001; Mitchell et al., 2001). This example describes phenotypic screens designed to identify genes for use as targets to ameliorate diseases in the areas of diabetes/metabolism, cardiology, neurology, opthalmology, reproductive biology, oncology and immunology/inflammation.

### 5.2 SATURATING THE DRUGGABLE GENOME

One of the advantages of doing genetic screens in the mouse model system is the ability to measure directly the physiological parameters relevant to disease. These direct measures allow the identification of gene products that, when modulated by small-molecule drugs, provide a therapeutic effect. This approach is supported by the excellent correlation between the knock-out phenotypes of the targets of marketed pharmaceutical drugs and the known efficacy and side-effects of those drugs (Zambrowicz and Sands, 2003). An example is a knock-out of the H+/K+ ATPase: the target of drugs such as Prilosec™ (AstraZeneca) used to lower gastric acid secretion for the treatment of gastric ulcer disease. Knock-out of either the alpha or beta subunit of ATPase results in animals with neutral stomach pH—a phenotype that correlates with the action of the pharmacological antagonists of ATPase (Scarff et al., 1999; Spicer et al., 2000). Similarly, mammalian screens can be set up to identify the genes that play a role in any specified therapeutic area. For instance, if one is interested in genes that may be important for the treatment of diabetes, it is possible to screen mutations in mice for direct effects on blood glucose and insulin levels, insulin sensitivity and other parameters such as obesity that play an important role in the diabetic process. There are genes involved in the regulation of glucose homeostasis, for example, the insulin receptor, which when mutated in mice results in animals with severe insulin resistance and frank diabetes (Accili et al., 1996; Joshi et al., 1996). Likewise, if one is interested in genes important for the treatment of osteoporosis, one can screen for mutations that increase or decrease bone mineral density, as observed for mice with mutations of the cathepsin K (Saftig et al., 1998) and osteoprotegerin genes (Bucay et al., 1998; Mizuno et al., 1998), respectively. This genetic approach is useful to analyze the role of a gene within the mammalian organism and its medical relevance.

The ability to measure parameters of mammalian physiology by screening in a mammalian organism stands in contrast to attempts to identify genes with disease relevance in lower model organisms such as Drosophila. The Drosophila system is useful for defining genetic pathways because of the ability to perform saturation screens for genetic modifiers of phenotypes that have been established already; yet, these genetic screens often are designed artificially and are removed from mammalian physiology. For instance, primary phenotypes to be used for modifier screens often are developed based upon overexpression, ectopic expression or expression of dominant or activated forms of a gene known to be involved in disease in the Drosophila eye (Therrien et al., 2000; Hirose et al., 2001; LaJeunesse et al., 2001; Schreiber et al., 2002; Sullivan and Rubin, 2002). Screens then are used to identify modifier genes that ameliorate or exacerbate the eye phenotype originally produced. These screens are able to elucidate genetic pathways and the types of genes that might play a role in a pathway of interest, but the corresponding mammalian genes still must be identified and tested for any relevance to the original disease or physiology of interest.

Saturation screens for genetic modifiers in non-mammalian organisms can provide clues for finding genes that may play a role in a disease-relevant pathway, but carrying out genetic screens directly in mammals for those genes is a preferred approach as more relevant to disease or physiology of interest. The question is whether the ability to scan a genome using saturation mutagenesis in invertebrate organisms outweighs the ability to screen directly in a more focused manner for genes that modulate disease-relevant mammalian physiology. Two of the challenges of conducting genetic screens in the mouse mammalian model have centered on the issues of the speed at which tractable genetic mutations can be generated and the large number of genes that must be processed to provide broad genomic coverage. While saturation modifier screens in mice need logistical support, it is possible to create mutations in all members of the so-called druggable classes of genes through gene targeting and gene trapping. This creates an opportunity to saturate the druggable mammalian genome, which is an important milestone in the evolution of drug discovery in the post-genome era. These druggable genes include secreted proteins that could be biotherapeutics themselves, potential targets for antibody-based therapeutics and small-molecule drug targets that belong to gene families that have proved themselves to be amenable to small molecule modulation based upon marketed drugs (Hopkins and Groom, 2002). The druggable genes include GPCRs (G-protein coupled receptors), ion channels, nuclear hormone receptors, key enzymes, kinases, proteases, secreted proteins and cell surface proteins. Indeed, one could argue that all mammalian disease or disease treatment pathways of interest probably contain druggable genes, so that by mutating all the druggable genes in the genome one can interrogate all pathways for points of therapeutic intervention.

Gene knock-out technologies were implemented on a large scale for saturation of the druggable genome. A program was instituted to knock out and analyze the resulting phenotypes for 5000 genes from the mammalian genome. The 5000 genes chosen are all members of druggable gene families. It was suggested that the druggable genome may be as small as about 3000 genes (Hopkins and Groom, 2002); therefore, screening 5000 genes is believed to be sufficient to saturate the mammalian druggable genome in order to identify those genes that have the greatest potential for disease treatment.

### 5.3 EFFECTIVELY SCREENING THE GENOME FOR NOVEL DRUG TARGETS

When generating knock-out mouse lines at a rate of 1000 per year, one needs a biological evaluation process that has a high probability of identifying potential drug targets, as assessed by the physiological consequences of gene disruption. A process that maximizes the potential to identify therapeutically significant genes was developed.

This screening process applies filters for genomic information. First, the genome is examined for members of druggable families. Second, knock-out mice are generated for selected genes at an average rate of 20 lines of mutant mice per week. A minimum cohort for initial evaluation is 16 animals; 8 Homozygous nulls, 4 Heterozygotes and 4 Wild-Type animals for each gene. This cohort size has produced reliable data from the primary screen upon which decisions for secondary screens can be made. This process involves the integration of bioinformatics, mouse genetics, robotics and high-speed physiological evaluation in an infrastructure with the ability to operate at the required rate. Generating, maintaining, genotyping and characterizing the required number of animals is logistically feasible.

An initial biological evaluation of the animals is a comprehensive clinical assessment of physiological parameters that can be measured effectively in high-throughput mode. Each test has relevance to one or more therapeutic areas and is designed to yield information that can be correlated directly with therapeutic intervention. This process includes an extensive battery of behavioral evaluations (neurology), blood pressure and heart rate measurements (cardiology) and a complete hematology survey supplemented with fluorescence-activated cell sorting (FACS) scans for immune function (immunology). The animals also are evaluated for body fat content, lean body mass (metabolism), bone mineral density, bone mineral content (endocrinology) and retinal integrity/vascularization (opthalmology). Effects on cell proliferation and reproductive organ development are studied (oncology) and fertility (reproductive biology) is assessed. This screening phase of biological investigation is referred to as Level 1 analysis.

This initial analysis of the physiological consequence of creating null mutations is designed to be unbiased with regard to potential outcome but to encompass phenotypes indicative of utility to our chosen therapeutic areas. All animals in all projects are submitted to the same tests in the same temporal sequence. This means that each test must be self-contained and have minimal impact on the outcome of subsequent tests. The aim of Level 1 analysis is to obtain a comprehensive understanding of gene function within the context of mammalian physiology. Variations from normal in any parameter are detected by comparison with cohort controls and with pooled historical data for all controls. Historical control data based on many animals give a good quantitative measure of normal values for each test and the level of background variation. Most of the tests are of primary importance to one particular therapeutic area (e.g. blood pressure and cardiology), but the total picture gained from this type of analysis is useful in identifying possible side-effects of target modulation. This allows the identification of targets with a potential for the development of target modulators.

In addition to therapeutic area-specific tests, multiple general diagnostic tests are performed. Level I pathology examines 52 tissues for the female and 53 tissues for the male. A complete gross necropsy is performed, with collection of tissues and photography of any significant gross lesions. Tissues are immersion-fixed in 10% neutral buffered formalin for 24 h, trimmed, processed to paraffin, embedded, sectioned at 4-5 mm, and stained with hematoxylin and eosin for histopathological examination. A board-certified pathologist examined tissues from one male and one female Homozygote for each project (Heterozygotes are examined for Homozygous lethal projects). Computer-assisted tomography (CAT) scanners operate effectively on mice and enable non-invasive evaluation of soft-tissue anatomy in addition to very refined skeletal analysis. Application of CAT (MicroCAT, ImTek Inc.) can be used to obtain morphological information non-invasively. All lesions are recorded and compared with controls in order to facilitate interpretation of phenotypes.

The output from all Level 1 tests is reduced to digital data and ported to a relational database. Data acquisition is rapid to the point that no Level 1 test is rate-limiting for the overall process. Numerical data is represented graphically with appropriate statistical tools, images are annotated by project scientists and interpretation of pharmaceutical relevance is summarized. It is therefore possible to gain a comprehensive view of the physiological function of every gene that is studied. This view encompasses those features that are most indicative of therapeutic potential in specific disease areas. Level 1 analysis has been a source of targets for drug discovery programs. Level 2 analysis entails the confirmation of Level 1 observations using additional animals and the application of specialized tests in a given project in reaction to Level 1 observations. Level 2 includes numerous therapeutic area-specific tests and challenge assays that cannot be used in the screening phase. Level 2 analysis may be triggered also through a hypothesis-driven approach. Level 3 analysis is designed for in-depth biological study in order to determine the merits of each target for assay development and high-throughput screening.

The decision to submit a given gene product to actual drug discovery is based on three criteria: modulation of the target by a small molecule, antibody or therapeutic protein could provide significant therapeutic effect with minimal or no discernable on-target side-effects; the target represents a potential breakthrough for the treatment of disease with significant advantages over existing therapies; and the program addresses a major unmet medical need. These criteria were applied to the analysis of multiple phenotypes and a number of projects were committed to further drug discovery following this analysis.

What follows is a brief description of the capabilities of the therapeutic area biology groups, including Level 1 and Level 2 tests that are most directly relevant to them.

### 5.4 HIGH-THROUGHPUT BIOLOGY: MAXIMIZING RETURN FROM REVERSE GENETICS

5.4.1 Endocrinology/Metabolism

Three of the most prevalent diseases of endocrinology/metabolism are Type TI diabetes, obesity and osteoporosis. A comprehensive panel of physiological tests was implemented for each disease process that have proved to provide reliable clinical descriptions of disease-related symptoms. These tests include measures of body composition index, glucose homeostasis and bone mass.

5.4.1.1 Level 1 Diabetes Tests

5.4.1.1.1 Glucose Tolerance Test

The glucose tolerance test (GTT) is the standard for defining impaired glucose homeostasis in mammals. For example, intraperitoneal glucose tolerance tests showed improved glucose clearance and the serum glucose and insulin levels were significantly lower in protein tyrosine phosphatase-1B (PTP-1B) and SHIP2 knock-out mice (Klaman et al., 2000; Clement et al., 2001). These findings indicate improved insulin sensitivity, a possibility that confirmed by hyperinsulinemic-euglycemic clamp studies in the PTP-1B knock-out mice (Klaman et al., 2000). These results suggest that these two proteins are targets for new therapeutics aimed at Type II diabetes. In addition, the ability of retinoid X receptor agonists to lower serum glucose and insulin levels is used as evidence that these agonists act as insulin sensitizers in vivo (Mukherjee et al., 1997). These examples validate the effectiveness of GTT for the identification of potential targets for diabetes. Glucose tolerance tests are performed using a Lifescan glucometer. Animals are injected i.p. with 2 g/kg D-glucose, delivered as a 20% solution, and blood glucose levels are measured at 0, 30, 60 and 90 min after injection (Klaman et al., 2000).

5.4.1.1.2 Urinalysis

Elevated glucose and/or ketone levels in urine are diagnostic markers for diabetes. Qualitative urinalysis is performed using Chemstrip 10 UA reagent strips (Roche) for the detection of glucose, bilirubin, ketones, blood, pH, protein, urobilinogen, nitrites and leukocytes in urine. Results are recorded using a Chemstrip 101 urine analyser.

5.4.1.1.3 Serum Insulin

Serum insulin levels are also diagnostic markers for diabetes. Insulin levels are assayed using a sensitive rat radioimmunoassay kit from Linco, which is sensitive to 0.02 ng/ml insulin in serum.

5.4.1.2 Level 2 Diabetes Tests

In Level 2, other tests are performed to verify and further define the role of targets in glucose homeostasis, for example, insulin tolerance test; insulin levels during GTT; insulin clearance (serum c-peptide/insulin ratio); measurement of serum free fatty acids, glycerol, glucagon, leptin, corticosterone; insulin content of pancreatic islets (radioimmunoassay); immunohistochemical analysis of pancreas for insulin, glucagon, somatostatin and pancreatic polypeptide; muscle and liver pathology, including glycogen and lipid content; pharmacological evaluation of liver slices, isolated soleus muscle and adipocytes.

5.4.1.3 Level 1 Obesity Tests

Animal weight and percent body fat are measured in Level 1 to identify obesity phenotypes.

5.4.1.3.1 Body Weight

All mice are weighed at 2, 4, 6, 8 and 16 weeks of age.

5.4.1.3.2 Dual-Energy X-Ray Absorptiometry

Dual-energy X-ray absorptiometry (DEXA) is used to identify increased total body fat in melanocortin-3 receptor knock-out mice (Butler et al., 2000) and decreased total body fat in melanin concentrating hormone 1 receptor knock-out mice; the latter observation was confirmed by direct analysis of fat pad weights (Marsh et al., 2002). Such results suggest these proteins as targets for novel obesity therapies. In addition, DEXA was used to show that the small-molecule insulin mimetic cpd2 blocks the accumulation of body fat in mice fed a high fat diet, an observation confirmed by direct analysis of fat pad weights (Air et al., 2002). A DEXA instrument (Lunar Piximus) is used to record bone mineral density, bone mineral content, percent body fat and total tissue mass (Nagy and Clair, 2000; Punyanitya et al., 2000). Although primarily aimed at metabolic and osteoporotic conditions, DEXA is a sensitive measure of all-round wellbeing and often contributes to diagnosis in other therapeutic areas.

5.4.1.4 Level 2 Obesity Tests

In Level 2, obesity targets are analyzed to determine whether they regulate metabolism, feeding, appetite or food absorption. Level 2 obesity tests include, for example, metabolic cages to measure food intake, water intake and fat malabsorption; Mini-Mitter telemetry for physical activity, core body temperature, drinking frequency and feeding frequency and duration; Oxymax measurement of metabolic rate and physical activity; home cage diet studies, including high-fat-diet challenge, food intake measurement and pair-feeding studies; fat mass by DEXA or nuclear magnetic resonance (Bruker Minispec); body composition analysis (analysis of carcass fat mass by Sohxlet; fat pad and organ weights); crosses to ob/ob mice; pharmacological challenge with leptin, melanocortin TI and neuropeptide Y; blood pressure.

5.4.1.5 Level 1 Osteoporosis Tests

5.4.1.5.1 Bone Microcomputed Tomography

Osteoporosis is characterized by a decreased bone mineral density due to a deficiency in bone production or increased bone absorption resulting in brittle bones. Specialized microcomputed tomography (micro-CT) machines have been developed with the capacity to provide quantitative and imaging data on the three-dimensional structure of mouse bones. This technique is used to demonstrate the efficacy of parathyroid hormone in a mouse model of osteoporosis (Alexander et al., 2001) and is used to describe in three dimensions the changes in bone resulting from the osteopetrotic mutation, which leads to osteopetrosis (Abe et al., 2000). A Scanco Medical mCT40 machine is used for measurements of bone mineral density. This machine permits visualization of trabecular bone structure, which is critical in evaluating overall bone quality. This is a much more sensitive analysis of bone than can be achieved using DEXA alone and is a specialized test for osteoporosis that we have implemented as part of our Level 1 analysis.

5.4.1.6 Level 2 Osteoporosis Tests

In Level 2, targets are analyzed to determine whether changes in bone mineral density are due to effects on bone deposition or bone resorption using various tests, for example, DEXA; micro-CT; undecalcified bone histomorphometry; bone histopathology; measurement of urinary helical peptide.

5.4.2 Cardiology

The major disease areas of interest in cardiology are hypertension, thrombosis, atherosclerosis and heart failure.

5.4.2.1 Level 1 Tests

5.4.2.1.1 Blood Pressure

Blood pressure measurements facilitate finding targets that, upon inhibition, lead to a reduction in blood pressure. Angiotensin-converting enzyme inhibitors and angiotensin receptor antagonists are effective drugs in the treatment of hypertension. Both knock-outs have low blood pressure. Blood pressure is measured using a non-invasive computerized tail-cuff system, the Visitech Systems BP-2000. This technique is a validated approach (Krege et al., 1995; Ito et al., 1995; Oliver et al., 1998; Sugiyama et al., 2001). Ten measurements of blood pressure are made per day on each of 4 days for each animal evaluated. Results are recorded as the pooled average of 40 measurements.

5.4.2.1.2 Zymosan Challenge Assay

Peritoneal leukocyte recruitment assays are used to identify targets that may regulate the inflammatory component of atherosclerosis. These assays detect abnormalities in immune cell recruitment to a site of inflammation. It has been shown in mutant such as C-C chemokine receptor 2 (CCR2) knock-outs that a defect in immune cell recruitment in these assays correlates well with a significant reduction in the inflammatory component of atherosclerosis and the subsequent plaque formation (Boring et al., 1997).

5.4.2.1.3 Blood Lipids

High cholesterol and triglyceride levels are recognized risk factors in the development of cardiovascular disease. Measuring blood lipids facilitates finding the biological switches that regulate blood lipid levels; inhibition of these switches should lead to a reduction in the risk for cardiovascular disease.

5.4.2.1.4 Optic Fundus Photography and Angiography

Optic fundus photography is performed on conscious animals using a modified Kowa Genesis small-animal-fundus camera (Hawes et al., 1999). Intraperitoneal injection of fluorescein permits the acquisition of direct light fundus images and fluorescent angiograms for each examination. In addition to direct opthalmological changes, this test can detect retinal changes associated with systemic diseases such as diabetes and atherosclerosis.

5.4.2.2 Level 2 Cardiology Tests

Level 2 cardiology tests include, for example, platelet aggregation; vascular injury by carotid cuff, chemically induced thrombosis; Poloxamer-induced atherosclerosis; aortic banding; permanent coronary occlusion; crosses with apolipoprotein E, low-density lipoprotein receptor and knock-outs.

5.4.3 Immunology

Our focus indications include acute inflammation, inflammatory bowel disease, transplantation, asthma, allergy, multiple sclerosis, rheumatoid arthritis and blood coagulation. The process of hematopoietic cell development and the regulation of mature immune cell function share several key signaling pathways, which are the result of similar molecular or cellular interactions. As an example, activation events via the antigen-specific T-cell receptor and co-stimulatory molecules are indispensable for both normal T cell development in the thymus and normal T-cell function during an immune response. Comprehensive phenotypic analysis of functionally relevant immune cell subpopulations in knock-out mice is essential for two reasons: it can reveal the role of a novel gene or expose the central role of a known gene in immune cell development and function; and at the same time it can provide the first hint about the potential mechanism that can lead to the observed immune deficiency.

5.4.3.1 Level 1 Tests

5.4.3.1.1 Complete Blood Cell Count

Evaluation of the cellular components of the immune system in knock-out mice and Wild-Type littermates is performed by automated determination of the absolute numbers of various cell types and ratios in the peripheral blood, i.e. complete blood cell count (CBC). This analysis is followed by a more detailed study using flow cytometry, which is designed to determine the relative proportions of CD4+ and CD8+ T cells, B cells, NK cells and monocytes in the mononuclear cell population. In the absence of a single molecular entity, disturbances in the proportion of any of the analyzed cell types could signal a key role for that molecule in governing the immune system, as exemplified in the following knock-out phenotypes.

The immunosuppressants cyclosporin A and FK506, which are useful to prevent transplant rejection, inhibit the immune response by inhibiting the catalytic activity of one or both isoforms of calcineurin A (can) in lymphocytes. Mice deficient in the b-isoform of the enzyme have a significant reduction in peripheral T lymphocytes due to 75% and 65% reductions in CD4+ and CD8+ positive thymocytes, respectively (Bueno et al., 2002). Mice deficient in expression of granulocyte colony-stimulating factor (G-CSF) exhibit chronic neutropenia with a 70-80% reduction in circulating neutrophils, whereas recombinant GCSF (Neupogen) stimulates neutrophil production and is used to treat neutropenia (Lieschke et al., 1994).

This test requires 135 μl of whole blood and employs a Cell-Dyn 3500R hematology analyzer. It reports on white blood cell count, neutrophils, lymphocytes, monocytes, eosinophils, basophils, red blood cell count and other standard hematology markers.

5.4.3.1.2 Blood Chemistry

A Cobas Integra 400 serum analyzer is used to measure a range of soluble serum components using approximately 85 ml of serum. Serum levels are recorded for alkaline phosphatase, albumin, total cholesterol, triglycerides, blood urea nitrogen, glucose, alanine aminotransferase, bilirubin, phosphate, creatinine, calcium and uric acid.

5.4.3.1.3 Fluorescence-Activated Cell Sorting (FACS)

Flow cytometry is designed to determine the relative proportions of CD4+ and CD8+ T cells, B cells, NK cells and monocytes in the mononuclear cell population. A Becton-Dickinson FACSCalibur 3-laser FACS machine is used to assess immune status. For Level 1 screening, this machine records CD4+/CD8−, CD8+/CD4−, NK, B cell and monocyte numbers, in addition to the CD4+/CD8+ ratio.

5.4.3.1.4 Ovalbumin Challenge

Chicken ovalbumin (OVA) is a T-cell-dependent antigen used as a model protein for studying antigen-specific immune responses in mice. It is non-toxic and inert and therefore will not cause harm to the animals even if no immune response is induced. The murine immune response to OVA has been well characterized, to the extent that the immunodominant peptides for eliciting T-cell responses have been identified. Anti-OVA antibodies are detectable 8-10 days after immunization using enzyme-linked immunosorbent assay, and determination of different isotypes of antibodies gives further information on the complex processes that may lead to a deficient response in genetically engineered mice.

The cyclosporin-mediated suppression of immune response once again demonstrates the similarity of phenotype using the suppressive agent or the genetic knock-out mice in this challenge model. Both cyclosporin-treated animals and mice knocked out for calcineurin A, in this case the a-isoform, show deficiency in T-cell-dependent antigen response (Puignero et al., 1995; Zhang et al., 1996).

Another example is the cytokine tumor necrosis factor a (TNF-a), whose important role in modulating inflammatory and antibody responses is well known. Two treatment options are available for patients with rheumatoid arthritis, a soluble receptor (Enbrel) and antibody (Remicade), both based on blocking the TNF-a activity. Underlining the effectiveness of drug therapy, mice deficient in TNF-a exhibit impaired humoral response to both T-cell dependent and T-cell-independent antigens (Pasparakis et al., 1996).

It is important to note that, even without antigenic challenge, the make-up of the immunoglobulin repertoire in a knock-out mouse is highly informative, because isotype switching of immunoglobulins is dependent on the interaction between B and T lymphocytes. Examples of the type of receptors required for normal function of T and B cells are the so-called co-stimulatory molecules, including CD28 and CD40 receptors, both of which are targets of antibody-based therapy with ongoing clinical trials for the treatment of various autoimmune diseases. In this case, mice deficient in either of these receptors register an impairment in immunoglobulin class switching, which is detectable in the serum of the animals (Shahinian et al., 1993, Kawabe et al., 1994). The protocol assesses the ability of mice to raise an antigen-specific immune response. Animals are injected i.p. with 50 mg of OVA emulsified in Complete Feund's Adjuvant; 8 days later the serum titer of anti-OVA antibodies (IgG1 and IgG2 subclasses) is measured.

5.4.3.2 Level 2 Immunology Tests

The following Level 2 tests are used to test for disease indication for a given target, for example, T-Cell activation, CD3 monoclonal antibody (mAb)+CD28 mAb induced; B-Cell activation, CD40 mAb+IL4 induced; mixed lymphocyte reaction provoked by irradiated BALB/C spleen cells; lipopolysaccharide challenge to evaluate acute phase response; Oxazolone sensitization and challenge for contact hypersensitivity; ovalbumin vaccine model; bovine collagen-induced arthritis; dextran sulfate gavage: inflammatory bowel disease model; ovalbumin+alum immunization followed by aerosol delivery of ovalbumin as asthma model; allograft rejection; blood coagulation assays: prothrombin time and activated partial thromboplastin; platelet aggregation; bone marrow transplantation.

5.4.4 Neurology

Neurology focuses on the identification of targets for anxiety, depression, schizophrenia, pain, sleep disorders, learning and memory disorders, neuromuscular disease and neurodegenerative disorders. The Level 1 assays have been based upon the behavioral phenotypes associated with knock-outs of known central nervous system targets as well as the actions of known drugs.

5.4.4.1 Level 1 Tests

5.4.4.1.1 Open Field Test

Several targets of known drugs have exhibited phenotypes in the open field test. These include knock-outs of the serotonin transporter, the dopamine transporter (Giros et al., 1996), and the GABA receptor (Homanics et al., 1997). An automated open-field assay is used to address changes related to affective state and exploratory patterns related to learning. First, the field (40×40 cm) is relatively large for a mouse, which is designed to pick up changes in locomotor activity associated with exploration. In addition, there are four holes in the floor to allow for nose-poking, an activity specifically related to exploration. Several factors have been designed to heighten the affective state associated with this test. The open-field test is the first experimental procedure in which the mice are tested, and the measurements taken are the subjects' first experience with the chamber. In addition, the open field is brightly lit. All these factors will heighten the natural anxiety associated with novel and open spaces. Thus, pattern and extent of exploratory activity, especially the center-to-total distance traveled ratio, may be able to discern changes related to susceptibility to anxiety or depression. A large arena (40 cm×40 cm, VersaMax animal activity monitoring system from AccuScan Instruments) with infrared beams at three different levels is used to record rearing, hole poke and locomotor activity. The animal is placed in the center and its activity is measured for 20 min. Data from this test are analyzed in five 4-min intervals. The total distance traveled (cm), vertical movement number (rearing), number of hole pokes and the center-to-total distance ratio are recorded.

5.4.4.1.2 Inverted Screen

This test is used to measure motor strength/coordination. Untrained mice are placed individually on top of a square (7.5 cm×7.5 cm) wire screen that is mounted horizontally on a metal rod. The rod is rotated 180° so that the mice are on the bottom of the screens. The following behavioral responses are recorded over a 1-min testing session: fell off, did not climb and climbed up.

5.4.4.1.3 Functional Observation Battery

This is a modified SHIRPA (Rogers et al., 2001) analysis in which the animals are scored systematically for 37 individual behavioral and physical characteristics, such as vision, response to touch, palpebral closure, etc. It is a formalization of the complete observation of the whole organism, which often gives the first hint as to phenotype.

5.4.4.1.4 Hot Plate and Formalin Paw

The 55° C. hot plate is a standard assay for measuring nociception in animals. Knock-out of either the m-opioid receptor (Sora et al., 1997) or COX 1 (Ballou et al., 2000) (both targets of analgesic drugs) results in effects on response latency in the hot-plate assay. Analgesia, such as that produced by morphine and other strong analgesics, is also detected using this assay. The hot-plate test is carried out by placing each mouse on a small, enclosed 55° C. hot plate (Hot Plate Analgesia Meter, Columbus instruments). Latency to a hind-limb response (lick, shake or jump) is recorded, with a maximum time on the hot plate of 30 s. Each animal is tested once.

The formalin paw assay is useful for hyperalgesia, as well as initial acute nociception. Recently, this assay has been automated and thus has become available for use in high-throughput analysis. Drugs that address novel mechanisms of hyperalgesia, without the side-effects of potent non-steroidal anti-inflammatory drugs, will be very useful new therapeutics.

5.4.4.1.5 Prepulse Inhibition

Prepulse inhibition is a pre-attentive process that has been shown to be deficient in patients with schizophrenia. This reduced ability to filter out environmental stimuli may contribute to both positive and negative symptoms of the disease. Antipsychotics can ameliorate some deficits in prepulse inhibition, therefore genetic inhibition of a target that can increase prepulse inhibition may presage a small-molecule therapeutic that can help patients with their disorder. The prepulse inhibition of the startle response assay is an automated measure of the startle response both with and without various intensities of prepulses. Targets whose genetic inhibition produces changes in prepulse inhibition without changes in the startle response itself may be excellent for the discovery of new therapeutics.

This test employs a San Diego Instruments SR-lab startle response system. Prepulse inhibition of the acoustic startle reflex occurs when a loud 120 decibel (dB) startle-inducing tone is preceded by a softer (prepulse) tone. The prepulse inhibition paradigm consists of six different trial types (70 dB background noise, 120 dB alone, 74+120 dB at postpartum day 4, 78+120 dB at postpartum day 8, 82+120 dB at postpartum day 12, and 90+120 dB at postpartum day 20) each repeated in pseudorandom order six times for a total of 36 trials. The maximum response to the stimulus (Vmax) is averaged for each trial type. The percentage inhibition of the animal's response to the startle stimulus is calculated for each prepulse intensity and then graphed. This test is being used increasingly as a model of human schizophrenia and a test for antipsychotic drugs.

5.4.4.1.6 Tail Suspension

The tail-suspension and forced-swim assays are two assays for the discovery and validation of novel antidepressants. The knock-out of the noradrenalin transporter, one target of the antidepressant Welbutrin™, demonstrates an increased struggle time in the tail-suspension assay (Xu et al., 2000). The tail-suspension assay used is automated, giving it added objectivity and making it appropriate for high-throughput analysis. Both of these assays measure the efforts of the subject to extricate itself from an inescapable situation, i.e. they measure a tendency toward ‘giving up’. Compounds known to reduce depressive symptoms in patients reduce the immobility time in tail suspension, therefore gene knock-outs that result in decreased time spent being immobile, in the absence of any general increase in activity levels (as measured in assays such as the open field), indicate targets for the discovery of novel therapeutics for the treatment of depression. In this particular set-up (PHM-300 Tail Suspension Test Cubicle) a mouse is suspended by its tail for 6 min, and in response the mouse will struggle to escape from this position. Extended struggle is taken as antidepressive behavior, whereas curtailed struggle is interpreted as depressive.

5.4.4.1.7 Circadian Rhythms

Changes in sleep patterns can be detected by examining activity continuously over a period of days and nights. An infrared beam system is used that monitors the horizontal locomotor activity of individual mice in their home cage environment for 3 days and nights. This facilitates obtaining an accurate indication of their sleep-wake cycle as well as overall locomotor activity rates. Changes in the normal circadian rhythm or an increase or decrease in the periods of activity during the normal sleep cycle indicates genes controlling sleep and indicates therapeutic utility for other conditions, such as depression or schizophrenia, in which normal sleep patterns are disrupted.

5.4.4.1.8 Trace Aversive Conditioning

Cognition, especially the loss of cognitive abilities in dementias such as Alzheimer's disease, later-stage Parkinson's and Huntington's disease, as well as in schizophrenia, is a major focus for drug discovery. This area has been hampered particularly by the lack of rapid assays that specifically target the learning and memory losses associated with these diseases, i.e. learning and memory dependent on areas of the brain such as the hippocampus. Assays generally used, such as the eight-arm radial arm maze or delayed-nonmatching-to-sample procedures require significant time and training. However, animals learn aversive conditioning very easily and this can be combined with ‘trace’ conditioning, in which there is a time interval between the signal stimulus and the aversive stimulus itself, to provide a rapidly (3-5 trials) learned response that is dependent upon the function of the hippocampus. As with most of our other assays, this assay has been automated to increase objectivity and make it appropriate for high throughput behavioral analysis. Gene knock-outs that affect learning and memory in this assay, without changes in basic sensory or motor function, will indicate targets for new treatments for cognitive disorders.

5.4.4.2 Level 2 Neurology Tests

Level 2 neurology tests include, for example, neurochemical analysis of dopamine, norepinephrine, serotonin and their primary metabolites in urine, blood, cerebrospinal fluid (CSF) and brain tissue; levels of melatonin and homocysteine in urine, blood, CSF and brain tissue; in situ hybridization/immunocytochemical analyses using Neo, LacZ or radioactivity; immunohistochemical analyses of markers of choice; pharmacological challenges in vivo; electroretinogram (vision); auditory brainstem response (hearing); detailed neuroanatomical/pathological analysis of brain, spinal cord, eye, ear and peripheral ganglia; field potential and whole-cell patch clamp in brain slices; whole-cell patch clamp of cultured neurons and other cells (HEK, etc.); fluorescence imaging of brain slices and cells; olfactory discrimination test (olfaction and social recognition); trace and delay aversive conditioning; social interaction and social recognition tests; zero maze (anxiety).

5.4.5 Oncology

The targets of oncology therapeutics fall into three major categories: cytotoxic agents such as DNA damaging agents or inhibitors of tubulin or topoisomerase, tissue-specific growth regulators such as estrogen receptor blockers and leutinizing hormone blockers, and disease-specific antitumor agents such as Gleevec™, Herceptin™ and Rituxan™. The oncology Level 1 screen is directed at targets for cancer drugs that fall into the same categories operating through control points in mammalian cell cycle, apoptosis or response to DNA damage.

5.4.5.1 Level 1 Tests

5.4.5.1.1 Embyronic Lethality and Reduced Viability

Targets for cytotoxic agents are identified first by embryonic lethality or reduced viability. These phenotypes are examined further to determine effects on cell cycle, apoptosis and angiogensis.

5.4.5.1.2 Tissue-Specific Growth Regulation

Targets affecting growth, differentiation and function of reproductive organs are examined through histopathologic survey of males, virgin females and lactating female mice.

5.4.5.1.3 Cell Proliferation

Oncogene targets that have a direct effect on cell cycle, DNA repair or apoptosis can manifest their function through changes in adult skin fibroblast proliferation. Punch biopsies are taken of skin samples from the backs of mutant mice and cohort controls. These are developed into primary fibroblast cultures and the fibroblast proliferation rates are measured in a controlled protocol. The ability of this assay to detect hyperproliferative and hypoproliferative phenotypes has been demonstrated with p53 and Ku80.

5.4.5.2 Level 2 Oncology Tests

Targets identified from Level 1 are characterized further for their role in mammalian tumorigenesis. Focus is placed on targets that are highly expressed in human tumor cell lines and capable of driving the tumor phenotype as demonstrated by gene knock-down studies or overexpression-driven tumorigenesis models in nude mice.

5.4.5.2.1 Quantitative Polymerase Chain Reaction for Analysis of Expression in cancerous and normal cell lines and tissues

Quantitative polymerase chain reaction of candidate genes is done using cDNA prepared from 66 cancer and nine normal cell lines from ATCC, seven primary cell strains from Clonetics, about three cancer lines and matched adjacent normal tissue controls from Ambion, MCF-7 breast cancer cells +/−17b-estradiol and LNCaP prostate cancer cells +/−dihydrotestosterone. This is done to identify targets that are overexpressed in cancerous cell lines relative to normal cell and tissue controls.

5.4.5.2.2 Gene Knock-Down Studies with Short Interfering RNA

Cancer cell lines determined to be overexpressing a target of interest are cotransfected with 3-6 short hairpin RNA vectors and blasticidin resistance vectors or synthetic short interfering RNAs to knock down the expression of specific targets. Assessment is made of the effects of RNA interference on in vitro proliferation, anchorage-dependent and anchorage-independent colony formation and the ability of cell lines to form tumors in nude mice.

5.4.5.2.3 Overexpression Studies for Putative Oncogenes

Oncology targets are tested to determine whether they can drive tumor formation. Full-length genes of interest are cloned into a mammalian expression vector and co-transfected into NIH3T3 and RK3E cells with a blasticidin-resistance vector. The resulting blasticidin-resistant polyclonal pools are tested in vitro for acquisition of anchorage independence, reduced serum dependence and increased focus-forming ability. Stably transfected cell lines expressing exogenous cDNAs of interest are then analyzed for their ability to form tumors in athymic nude mice.

### 5.5 CONCLUSIONS

Described here is a new conceptual framework for the discovery of drugs using the mammalian genome as a starting point in the analysis. The framework requires genetic antagonism of the drug target combined with a comprehensive in vivo physiological characterization of target function before any chemical screens for pharmaceutical agents are launched. This process constitutes a powerful genetic screen for the targets that allow for maximizing therapeutic effects while minimizing side-effects resulting from therapies modulating the target. In addition, determination of the role of the target in mammalian physiology enables identification of the medical indications for the therapeutics to be developed. Although this may appear an obvious prerequisite, it is important to note that many screens are conducted today against molecular targets for which the medical utility is either completely unknown or hypothesized based on only biochemical, gene expression or lower model organism data.

The mammalian genetic screen described here is engineered specifically to reveal those genes that encode control points in physiology useful in the treatment of major diseases. The tests described here measure important medical parameters of physiology that are associated with points of therapeutic intervention and medical needs. Additionally, the tests are robust in their application to thousands of animals.

The screen used to identify therapeutic targets can be applied again to demonstrate the efficacy and potential side-effects of candidate therapeutic agents. This broad phenotypic screen, guided by mammalian genetics, provides a new performance level for the preclinical testing of compounds that are developed to interact with chosen targets. The screen enables identification of the key biomarker indicators of efficacy that should be followed when a compound is at the first-time-in-mammal stage. The genetic tools available for preclinical studies include not only Wild-Type animals but also knock-outs and knock-ins containing the actual human gene targets. The knock-out animals provide guidance for determining the efficacy of novel therapeutic agents. Another powerful aspect of the preclinical testing capabilities includes the treatment of knock-out animals themselves with compounds specific for the target. In such a scenario any effects seen, outside those associated with the knock-out state, are, by definition, off-target side-effects attributable to the compound itself. The ability to manipulate the mouse genome at will provides a powerful tool to define accurately the on-target versus off-target side-effects produced by a given agent. Such new approaches are also very useful in combination with medicinal chemistry strategies to optimize therapeutic agents.

In the post-genome era, a systematic in vivo screen for targets is a precondition for high-throughput screening of small molecule therapeutics to identify the targets for treatments for disease.

### 5.6 REFERENCES

Abdelilah, S., et al. (1996) Development 123, 217-227.
Abe, S., et al. (2000) Br. J. Radiol. 73, 1078-1082.
Abuin, A., et al. (2002) Trends Biotechnol. 20, 36-42.
Accili, D., et al. (1996) Nat. Genet. 12, 106-109.
Air, E. L., et al. (2002) Nat. Med. 8, 179-183.
Alexander, J. M., et al. (2001) J Bone Miner. Res. 16, 1665-1673.
Baier, H., et al. (1996) Development 123, 415-425.
Ballou, L. R., et al. (2000) Proc. Natl. Acad. Sci. USA 97, 10272-10276.
Boring, L., et al. (1997) J. Clin. Invest. 100, 2552-2561.
Brand, M., et al. (1996) Development 123, 129-142.
Brockerhoff, S. E., et al. (1995) Proc. Natl. Acad. Sci. USA 92, 10545-10549.
Bucay, N., et al. (1998) Genes Dev. 12, 1260-1268.
Bueno, O. F., et al. (2002) Proc. Natl. Acad. Sci. USA 99, 9398-9403.
Butler, A. A., et al. (2000) Endocrinology 141, 3518-3521.
Chalfie, M., et al. (1981) Cell 24, 59-69.
Chen, J. N., et al. (1996) Development 123, 293-302.
Clement, S., et al. (2001) Nature 409, 92-97.
Giros, B., et al. (1996) Nature 379, 606-612.
Golling, G., et al. (2002) Nat. Genet. 31, 135-140.
Granato, M., et al. (1996) Development 123, 399-413.
Haffter, P., et al. (1996) Development 123, 1-36.
Hawes, N. L., et al. (1999) Mol. Vis. 5, 22.
Hedgecock, E. M., et al. (1983) Science 220, 1277-1279.
Hirose, F., et. al. (2001) Mol. Cell Biol. 21, 7231-7242.
Homanics, G. E., et al. (1997) Proc. Natl. Acad. Sci. USA 94, 4143-4148.
Hopkins, A. L. and Groom, C. R. (2002) Nat. Rev. Drug Discov. 1, 727-730.
Horvitz, H. R. and Sulston, J. E. (1980) Genetics 96, 435-454.
Ito, M., et al. (1995) Proc. Natl. Acad. Sci. USA 92, 3521-3525.
Joshi, R. L., et al. (1996) EMBO J. 15, 1542-1547.
Kawabe, T., et al. (1994) Immunity 1, 167-178.
Kelsh, R. N., et al. (1996) Development 123, 369-389.
Klaman, L. D., et al. (2000) Mol. Cell Biol. 20, 5479-5489.
Krege, J. H., et al. (1995) Hypertension 25, 1111-1115.
LaJeunesse, D. R., et al. (2001) Genetics 158, 667-679.
Leighton, P. A., et al. (2001) Nature 410, 174-179.
Lieschke, G. J., et al. (1994) Blood 84, 1737-1746.
Malicki, J., et al. (1996a) Development 123, 263-273.
Malicki, J., et al. (1996b) Development 123, 275-283.
Marsh, D. J., et al. (2002) Proc. Natl. Acad. Sci. USA 99, 3240-3245.
Mitchell, K. J., et al. (2001) Nat. Genet. 28, 241-249.
Mizuno, A., et al. (1998) Biochem. Biophys. Res. Commun. 247, 610-615.
Mukherjee, R., et al. (1997) Nature 386, 407-410.
Mullins, M. C., et al. (1994) Curr. Biol. 4, 189-202.
Nagy, T. R. and Clair, A. L. (2000) Obes. Res. 8, 392-398.
Neuhauss, S. C., et al. (1996) Development 123, 357-367.
Nusslein-Volhard, C. and Wieschaus, E. (1980) Nature 287, 795-801.
Odenthal, J., et al. (1996) Development 123, 103-115.
Oliver, P. M., et al. (1998) Proc. Natl. Acad. Sci. USA 95, 2547-2551.
Pasparakis, M., et al. (1996) J. Exp. Med. 184, 1397-1411.
Piotrowski, T., et al. (1996) Development 123, 345-356.
Puignero, V., et al. (1995) Int. Arch. Allergy Immunol. 108, 142-147.
Punyanitya, M., et al. (2000) FASEB J. 14, 497.
Rogers, D., et al. (2001) Neurosci. Lett. 306, 89-92.
Saftig, P., et al. (1998) Proc. Natl. Acad. Sci. USA 95, 13453-13458.
Scarff, K. L., et al. (1999) Gastroenterology 117, 605-618.
Schier, A. F., et al. (1996) Development 123, 165-178.
Schreiber, S. L., et al. (2002) Genesis 33, 141-152.
Shahinian, A., et al. (1993) Science 261, 609-612.
Solnica-Krezel, L., et al. (1996) Development 123, 67-80.
Sora, I., et al. (1997) Proc. Natl. Acad. Sci. USA 94, 1544-1549.
Spicer, Z., et al. (2000) J. Biol. Chem. 275, 21555-21565.
Stemple, D. L., et al. (1996) Development 123, 117-128.
Sugiyama, F., et al. (2001) Genomics 71, 70-77.
Sullivan, K. M. and Rubin, G. M. (2002) Genetics 161, 183-193.
Therrien, M., et al. (2000) Genetics 156, 1231-1242.
Walke, D. W., et al. (2001) Curr. Opin. Biotechnol. 12, 626-631.
Wiles, M. V., et al. (2000) Nat. Genet. 24, 13-14.
Xu, F., et al. (2000) Nat. Neurosci. 3, 465-471.
Zambrowicz, B. P. and Sands, A. T. (2003) Nat. Rev. Drug Discov. 2, 38-51.
Zambrowicz, B. P., et al. (1998) Nature 392, 608-611.
Zhang, B. W., et al. (1996) J. Exp. Med. 183, 413-420. 278.

### Example 6

### A Mouse Library with 6 Distinct Mouse Mutants

A mouse library of 6 distinct mouse mutants or mouse lines was generated. The following table (Table 1) describes the vector used to generate each line. Each vector is a gene trap vector that may integrate into a gene in the mouse genome and result in a gene transcript comprising vector sequences and exon sequences from the gene. The polynucleotide sequences referenced in Table 1, and disclosed in the appended Sequence Listing, were obtained by sequencing transcripts that include sequences of the gene trap vector. Table 1 also provides phenotypic data of each mouse line.

TABLE 1

| Line | | SEQ | |
|---|---|---|---|
| No. | Vector: | ID: | Phenotype: |
| 1. | VICTR48 | 9 | Mutation of a hypothetical mouse gene (1700061G19Rik) resulted |
| | | | in enhanced learning/memory in female (−/−) mice. Transcript was |
| | | | absent by RT-PCR. |
| 2. | VICTR48 | 10 | Mutation of the gene encoding the ortholog of human |
| | | | immunoglobulin superfamily, member 9 (IGSF9) resulted in |
| | | | increased cholesterol levels in (−/−) mice. Transcript was absent by |
| | | | RT-PCR. |
| 3. | VICTR48 | 11 | Mutation of the gene encoding the ortholog of a human |
| | | | hypothetical kinase resulted in hydrocephalus, cerebellar cortical |
| | | | dysplasia, and neurological and ophthalmological abnormalities in |
| | | | (−/−) mice. Male (−/−) mice exhibited impaired glucose tolerance. |
| | | | Transcript was absent by RT-PCR. |
| 4. | VICTR48 | 12 | Mutation of the gene encoding the ortholog of human NAD kinase |
| | | | (FLJ13052) resulted in lethality of (−/−) mutants. Male (+/−) mice |
| | | | exhibited increased serum insulin and impaired glucose tolerance. |
| | | | The (+/−) mice also exhibited decreased MCP-1, TNF-alpha, and |
| | | | IL-6 responses to LPS challenge. |
| 5. | VICTR48 | 13 | Mutation of the gene encoding the ortholog of human procollagen |
| | | | C-endopeptidase enhancer 2 (PCOLCE2) resulted in growth |
| | | | retardation in male (−/−) mice. Transcript was absent by RT-PCR. |
| 6. | VICTR48 | 14 | Mutation of the gene encoding the ortholog of a human |
| | | | hypothetical leucine-rich repeat protein (MGC25027) resulted in a |
| | | | decreased inflammatory response in (−/−) mice. Transcript was |
| | | | absent by RT-PCR. |

The polynucleotide sequences referenced in Table 1 were used to search the Genbank database to identify mouse polynucleotide sequences by accession numbers (Mouse Nucleotide Accession Nos.) that represent the full length transcript corresponding to the gene carrying the engineered mutation in each mouse line of Table 1. Also obtained were the corresponding polypeptide sequences by accession numbers (Mouse Protein Accession Nos.). Also obtained were the human polynucleotide (Human Nucleotide Accession Nos.) and polypeptide sequences (Human Protein Accession Nos.) that correspond to the mouse sequences. The Genbank accession numbers for all four types of sequences are shown below in Table 2 for each of the 6 mouse lines.

TABLE 2

| | Mouse | | | |
|---|---|---|---|---|
| | Nucleotide | Mouse Protein | Human Nucleotide | Human Protein |
| Line No. | Accession Nos.: | Accession Nos.: | Accession Nos.: | Accession Nos.: |
| 1. | AK028078 | XP_140052 | N/A | N/A |
| 2. | NM_033608 | NP_291086 | NM_020789 | Q9P2J2 |
| 3. | NM_033608 | NP_291086 | NM_020789 | Q9P2J2 |
| 4. | NM_010497 | O88844 | NM_005896 | O75874 |
| 5. | NM_029620 | NP_083896 | NM_013363 | NP_037495 |
| 6. | NM_177303 | P59383 | NM_152611 | Q5JWV6 |

### Example 7

### Polynucleotides Sequences Obtained From Hybrid Transcripts

Gene trap vectors of the current invention were introduced into ES cells. A selection protocol was employed to identify cells with a copy of a gene trap vector integrated into their genome. Hybrid transcripts were identified and isolated from the cells following selection for sequencing of non-vector sequences included in those hybrid transcripts, i.e., sequences from the mouse genome and trapped in the hybrid transcript (trapped sequences). These assays were carried our as discussed herein and in United States Patent Application Number 20050059060, which is incorporated herein for that and any other purpose. Each trapped sequence obtained is believed to represents at least a part of a gene or an entire gene that is expressed in ES cells and/or other cell types. Thus, each trapped sequence is believed to be a sequence tag or sequence identifier to identify a gene in a mouse, or another species that is sufficiently closely related to mouse. Trapped sequences are shown in SEQ ID NOS. 15 to 3423.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein, will become apparent to those skilled in the art from the foregoing description. Such modifications are intended to fall within the scope of the appended claims. All cited publications, patents, and patent applications are herein incorporated by reference in their entirety for any purpose.

1-4. (canceled)

7. A transgenic mouse whose genome comprises a disruption of a gene comprising SEQ ID NO: 15 and wherein said disruption results in a phenotype that differs from that of a wild-type mouse.
